(19) United States
(12) Patent Application Publication
(10) Pub. No.: US 2011/0015137 A1
(43) Pub. Date: Jan. 20, 2011

(54) RECA INHIBITORS AND THEIR USES AS MICROBIAL INHIBITORS OR POTENTIATORS OF ANTIBIOTIC ACTIVITY

(75) Inventor: Guillaume Cottarel, Mountain View, CA (US)

Correspondence Address: CHOATE, HALL & STEWART LLP, TWO INTERNATIONAL PLACE, BOSTON, MA, 02110, US

(73) Assignee: Trustees of Boston University, Boston, MA (US)

(21) Appl. No.: 12/667,725

(22) PCT Filed: Jul. 7, 2008
(86) PCT No.: PCT/US08/69343

§ 371 Date: Sep. 22, 2010

Related U.S. Application Data
(60) Provisional application No. 60/958,432, filed on Jul. 5, 2007.

(51) Int. Cl. A61K 38/12; A61K 31/496; A61K 31/7036; A61K 31/704; A61K 31/437; A61K 31/135; A61K 31/47; A61K 31/05; A61K 31/473; A61K 31/536; C07D 413/14; A61P 31/04

(52) U.S. Cl. 514/21.1; 514/253.08; 514/36; 514/34; 514/285; 514/656; 514/313; 514/736; 514/297; 514/229.8; 544/102

(57) ABSTRACT

The present invention provides RecA inhibitors, compositions containing them, systems for identifying or characterizing them, and methods of using them.

### RELATED APPLICATIONS

This application claims priority under 35 U.S.C. §119(e) to U.S. Provisional Application U.S. Ser. No. 60/958,432 filed on Jul. 5, 2007, which is incorporated herein by reference in its entirety.

This invention was made with Government Support under Contract No. EF-0425719 awarded by the National Science Foundation. The Government has certain rights in the invention.

### BACKGROUND OF THE INVENTION

Emerging resistance to antibiotics and the poor pipeline of new anti-bacterial compounds has created an urgent unmet need for treating infectious diseases that constitutes a significant threat to international public health and national biodefense. For example, the CDC currently recommends a 90 day course of ciprofloxacin for treatment of victims suspected of being infected with Bacillus anthracis spores. For suspected infection with Yersinia pestis, Francisella tularensis, and Burkholderia and Brucella species, a number of different antibiotics are recommended. However, strains of these species resistant to these treatments have been observed. To overcome drug-resistance, therapies currently under development include multiple antibiotic combinations and inhibitors of efflux pumps. However, combinations of existing antimicrobials would be ineffective against bacteria engineered to be resistant to standard antimicrobials of each class, and the development of safe and effective inhibitors of multiple efflux pumps has thus far been elusive. Furthermore, a number of potent antibiotic agents have been found to be too toxic for clinical use or to have significant side effects that limit their therapeutic utility.

Therefore, there is a need for new strategies to overcome drug resistance and for new approaches to antibiotic drug discovery.

### SUMMARY OF THE INVENTION

The present invention provides novel and inventive strategies to overcome antimicrobial resistance. The present invention encompasses the recognition that RecA is a virulence factor and a stand-alone target, and that RecA inhibitors are uniquely useful as potentiators of anti-infective activity or as stand-alone anti-infective compounds.

According to the present invention, RecA inhibitors may be used alone as antibiotics, may be used in combination with one or more other agents with antibiotic activities, or both. When RecA inhibitors are administered in combination with one or more such antibiotic agents, the antibiotic agent(s) may often be utilized at a lower dose, and/or less frequent dosing regimen than their conventional dose and/or schedule. In certain embodiments, this is employed to reduce the antibiotic(s)'s toxicity. When a RecA inhibitor is used in combination with an antibiotic agent, the RecA inhibitor and antibiotic agent may be administered substantially simultaneously using the same or different routes of administration. Alternatively, the RecA inhibitor and antibiotic agent may be administered sequentially, using the same or different routes of administration. In certain embodiments, the RecA inhibitor is administered prior to administration of the antibiotic agent.

In some embodiments, inventive RecA inhibitors have broad spectrum activity in that they inhibit one or more activities of RecA (or its relevant homologs) from a wide range of different organisms (i.e., at least two different organisms or organisms from two different families). In other embodiments, inventive RecA inhibitors have a narrow spectrum activity in that they inhibit one or more activities of RecA (or its relevant homologs) from a specific family of organisms or from a specific organism. In certain embodiments, inventive RecA inhibitors inhibit one or more activities of RecA (or its relevant homologs) from a disease-causing organism (in particular an organism that causes disease in a mammal, e.g., a human). In some embodiments, however, the RecA inhibitors (which may be broad spectrum with regard to microbes) do not inhibit RecA (or relevant RecA homologs) from one or more higher organisms (e.g., mammals, humans).

In some embodiments, RecA inhibitors of the present invention have a structure depicted in any one of FIGS. 1 and 2, or a derivative thereof. In some embodiments, RecA inhibitors of the present invention have a pharmacophore structure depicted in FIG. 7, or a pharmacophore structure substantially similar thereto. In some embodiments, RecA inhibitors of the present invention is actinomycin D or a derivative thereof (e.g., a structure depicted in FIG. 6).

In some embodiments, RecA inhibitors in accordance with the present invention has the following structure:

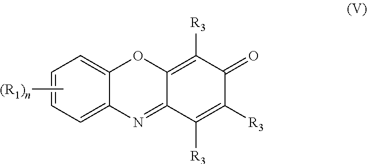

wherein
n is an integer between 0 and 4; inclusive;
each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —OC(O)R_{A}; —OC(O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; —OC(O)R_{C}; —OC(O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

Among other things, the present invention provides RecA inhibitors, and methods and systems for identifying, making, characterizing, and/or using RecA inhibitors. The present invention further provides methods of developing, designing or predicting novel compounds with similar or improved RecA inhibitory activity based on RecA inhibitors identified herein.

### BRIEF DESCRIPTION OF THE DRAWING

FIG. 1 depicts exemplary structures of RecA inhibitors identified from a high-throughput RecA-luciferase assay.

FIG. 2 depicts additional exemplary structures of RecA inhibitors identified from a high-throughput RecA-luciferase assay.

FIG. 3 illustrates exemplary results from an in vitro RecA ATPase assay confirming the RecA inhibitory activity of actinomycin D.

FIG. 4 illustrates exemplary results from a cell growth assay demonstrating that actinomycin D potentiates the anti-microbial activity of gentamycin.

FIG. 5 illustrates exemplary results from a cell growth assay demonstrating that actinomycin D potentiates the anti-microbial activity of ciprofloxacin.

FIG. 6 presents exemplary structures of actinomycin D analogues.

FIG. 7 presents exemplary pharmacophore structures of RecA inhibitors as shown in FIGS. 1 and 2.

FIG. 8 depicts the core structures and exemplary numbering system of classical quinolone antibiotics (4-quinolone and 4-naphthyridine systems).

FIG. 9 illustrates core structures of quinolone antibiotics related to the 4-oxo-1,4-dihydroquinoline and 4-oxo-1,4 dihydronapthyridine systems

FIG. 10, Panels A and B depict particular core structures of certain quinolone antibiotics.

FIG. 11 is a schematic diagram showing functional modules of the E. coli RecA protein Amino acid numbers bracketing modules associated with particular functional activities are shown. These modules are highly conserved among bacteria. The figure is taken from Karlin & Bricchiere, J. Bacteriol. 178:1881, 1996, and is not drawn to scale.

### DEFINITIONS

Definitions of specific functional groups and chemical terms in connection with chemical compounds are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl(p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS),1,3-(1,1,3,3-tetraisopropyldisiloxanylidene)derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine(Tr), N-[(4-methoxyphenyl)diphenylmethyl]amine(MMTr), N-9-phenylfluorenylamine(PhF), N-2,7-dichloro-9-fluorenylmethyleneamine, N-ferrocenylmethylamino(Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide(Dpp), dimethylthiophosphinamide(Mpt), diphenylthiophosphinamide(Ppt), dialkyl phosphoramidates, dibenzyl phosphoramidate, diphenyl phosphoramidate, benzenesulfenamide, o-nitrobenzenesulfenamide(Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide(Ts), benzenesulfonamide, 2,3,6-trimethyl-4-methoxybenzenesulfonamide(Mtr), 2,4,6-trimethoxybenzenesulfonamide(Mtb), 2,6-dimethyl-4-methoxybenzenesulfonamide(Pme), 2,3,5,6-tetramethyl-4-methoxybenzenesulfonamide(Mte), 4-methoxybenzenesulfonamide(Mbs), 2,4,6-trimethylbenzenesulfonamide(Mts), 2,6-dimethoxy-4-methylbenzenesulfonamide(iMds), 2,2,5,7,8-pentamethylchroman-6-sulfonamide(Pmc), methanesulfonamide(Ms), β-trimethylsilylethanesulfonamide (SES),9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl(propargyl), 1-propynyl, and the like.

The term “alkoxy”, or “thioalkyl” as used herein refers to an alkyl group, as previously defined, attached to the parent molecule through an oxygen atom or through a sulfur atom. In certain embodiments, the alkyl, alkenyl, and alkynyl groups contain 1-20 alipahtic carbon atoms. In certain other embodiments, the alkyl, alkenyl, and alkynyl groups contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of alkoxy, include but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “alkylamino” refers to a group having the structure —NHR′, wherein R′ is aliphatic, as defined herein. In certain embodiments, the aliphatic group contains 1-20 aliphatic carbon atoms. In certain other embodiments, the aliphatic group contains 1-10 aliphatic carbon atoms. In yet other embodiments, the aliphatic group employed in the invention contain 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each an aliphatic group, as defined herein. R and R′ may be the same or different in an dialkyamino moiety. In certain embodiments, the aliphatic groups contains 1-20 aliphatic carbon atoms. In certain other embodiments, the aliphatic groups contains 1-10 aliphatic carbon atoms. In yet other embodiments, the aliphatic groups employed in the invention contain 1-8 aliphatic carbon atoms. In still other embodiments, the aliphatic groups contains 1-6 aliphatic carbon atoms. In yet other embodiments, the aliphatic groups contains 1-4 aliphatic carbon atoms. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R_{X}; —CO₂(R_{X}); —CON(R_{X})₂; —OC(O)R_{X}; —OCO₂R_{X}; —OCON(R_{X})₂; —N(R_{X})₂; —S(O)₂R_{X}; —NR_{X}(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term “heteroaryl”, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R_{X}; —CO₂(R_{X}); —CON(R_{X})₂; —OC(O)R_{X}; —OCO₂R_{X}; —OCON(R_{X})₂; —N(R_{X})₂; —S(O)₂R_{X}; —NR_{X}(CO)R_{X}, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic, or heterocyclic moieties, may optionally be substituted with substituents including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R_{X}; —CO₂(R_{X}); —CON(R_{X})₂; —OC(O)R_{X}; —OCO₂R_{X}; —OCON(R_{X})₂; —N(R_{X})₂; —S(O)₂R_{X}; —NR_{X}(CO)R_{X}, wherein each occurrence of R_{X} independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R_{X}; —CO₂(R_{X}); —CON(R_{X})₂; —OC(O)R_{X}; —OCO₂R_{X}; —OCON(R_{X})₂; —N(R_{X})₂; —S(O)₂R_{X}; —NR_{X}(CO)R_{X}, wherein each occurrence of R_{X} independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R_{X}; —CO₂(R_{X}); —CON(R_{X})₂; —OC(O)R_{X}; —OCO₂R_{X}; —OCON(R_{X})₂; —N(R_{X})₂; —S(O)₂R_{X}; —NR_{X}(CO)R_{X}, wherein each occurrence of R_{X} independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

The term “carbocycle,” as used herein, refers to an aromatic or non-aromatic ring in which each atom of the ring is a carbon atom.

The term “independently selected” is used herein to indicate that the R groups can be identical or different.

As used herein, the term “labeled” is intended to mean that a compound has at least one element, isotope, or chemical compound attached to enable the detection of the compound. In general, labels typically fall into three classes: a) isotopic labels, which may be radioactive or heavy isotopes, including, but not limited to, ²H, ³H, ³²P, ³⁵S, ⁶⁷GA, ^{99m}Tc (Tc-99m), ¹¹¹In, ¹²³I, ¹²⁵I, ¹⁶⁹Yb and ¹⁸⁶Re; b) immune labels, which may be antibodies or antigens, which may be bound to enzymes (such as horseradish peroxidase) that produce detectable agents; and c) colored, luminescent, phosphorescent, or fluorescent dyes. It will be appreciated that the labels may be incorporated into the compound at any position that does not interfere with the biological activity or characteristic of the compound that is being detected. In certain embodiments, hydrogen atoms in the compound are replaced with deuterium atoms (2H) to slow the degradation of a compound in vivo. In certain embodiments of the invention, photoaffinity labeling is utilized for the direct elucidation of intermolecular interactions in biological systems. A variety of known photophores can be employed, most relying on photoconversion of diazo compounds, azides, or diazirines to nitrenes or carbenes (See, Bayley, H., Photogenerated Reagents in Biochemistry and Molecular Biology (1983), Elsevier, Amsterdam.), the entire contents of which are hereby incorporated by reference. In certain embodiments of the invention, the photoaffinity labels employed are o-, m- and p-azidobenzoyls, substituted with one or more halogen moieties, including, but not limited to 4-azido-2,3,5,6-tetrafluorobenzoic acid.

The term “unsaturated,” as used herein, means that a moiety has one or more units of unsaturation.

Antibiotic agent: The term “antibiotic agent” refers to an agent that inhibits and/or stops growth and/or proliferation of one or more species of microorganism (e.g., bacteria or fungus). An antibiotic agent may display activity in vitro (e.g., when contacted with cells in culture), in vivo (e.g., when administered to a subject at risk of or suffering from an infection), or both. The term “antibiotic agent” may be used to refer to bactericidal agents (i.e., agents that kill bacteria) and/or bacteriostatic agents (i.e., agents that inhibit or stop bacterial growth or proliferation but does not kill the cells).

Conventional dose: A “conventional dose” of an antibiotic agent means a dose that is (i) in the case of humans or animals, approved by a regulatory body such as, for example, the United States Food and Drug Administration; (ii) recommended on the package insert; (iii) in the case of humans, recommended in Goodman and Gilman, supra; Katzung, supra, and/or The Merck Manual of Diagnosis and Therapy, 17^{th} ed. (1999), or the 18^{th} ed (2006) following its publication, Mark H. Beers and Robert Berkow (eds.), Merck Publishing Group; and/or (iv) in the case of animals, recommended in The Merck Veterinary Manual, 9^{th} ed., Kahn, C. A. (ed.), Merck Publishing Group, 2005. It will be appreciated that a conventional dose may be modified appropriately for an individual subject taking into account, for example, factors such as the subject's age, diet, renal and/or hepatic function, other medications, other diseases or conditions (i.e., diseases or conditions other than the infection for which an antibiotic agent is administered), past experience with the antibiotic agent, etc.

Derivatives or analogues: As used herein, a derivative or an analogue refers to a compound formed from or designed to arise from another compound. Typically, a derivative or an analogue of a compound is formed or can be formed by replacing at least one atom with another atom or a group of atoms. As used in connection with the present invention, a derivative or an analogue of a compound is a modified compound that shares on eor more chemical characteristics or features that are responsible for the activity of the compound. In some embodiments, a derivative or an analogue of a compound has a pharmacophore structure of the compound (see the definition of pharmacophore below). In some embodiments, a derivative or an analogue of a compound has a pharmacophore structure of the compound with at least one side chain or ring linked to the pharmacophore that is present in the original compound (e.g., a functional group). In some embodiments, a derivative or an analogue of a compound has a pharmacophore structure of the compound with side chains or rings linked to the pharmacophore substantially similar to those present in the original compound. As used herein, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical linkage bonds (e.g., rotatable linkage bonds). In some embodiments, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical atom coordinates defining the structures, or equivalent structures having a root mean square of deviation less than about 5.0 Å (e.g., less than about 4.5 Å, less than about 4.0 Å, less than about 3.5 Å, less than about 3.0 Å, less than about 2.5 Å, less than about 2.0 Å, less than about 1.5 Å, or less than about 1.0 Å). In some embodiments, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical atom coordinates defining surface-accessible features (e.g., hydrogen bond donors and acceptors, charged/ionizable groups, and/or hydrophobic patches), or equivalent features having a root mean square of deviation less than about 5.0 Å (e.g., less than about 4.5 Å, less than about 4.0 Å, less than about 3.5 Å, less than about 3.0 Å, less than about 2.5 Å, less than about 2.0 Å, less than about 1.5 Å, or less than about 1.0 Å). As used herein, a derivative or an analog of a compound is not the compound itself.

Effective amount: In general, an “effective amount” of a biologically and/or pharmacologically active agent is an amount sufficient to achieve a desired biological and/or pharmacological effect when delivered to a cell or organism according to a selected administration form, route, and/or schedule. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses.

For example, an effective amount of an antibiotic agent may be an amount sufficient to achieve one or more of the following: (i) inhibit microbial growth in culture or in vivo; (ii) reduce the severity of or prevent one or more symptoms or signs of an infection; (iii) significantly reduce the risk of recurrence of an infection; (iv) significantly reduce the risk of a clinically significant infection in a subject who has been exposed to an infectious agent, etc.

Comparably, an effective amount of a potentiating agent may be an amount sufficient to achieve the same level of antibiotic activity with a particular antibiotic agent as is achieved when that antibiotic agent is administered at its conventional dose, in circumstances where the antibiotic agent is administered at a reduced dose as compared with its conventional dose.

An effective amount of a RecA inhibitor according to the present invention may be, for example, (i) an amount sufficient to act as an antibiotic agent; (ii) an amount sufficient to inhibit one or more activities of RecA (or a relevant homolog); (iii) an amount sufficient to potentiate activity of one or more antibiotic agents (e.g., with which the RecA inhibitor is administered in combination); and/or (iv) an amount sufficient to reduce the incidence of resistance developed to another antibiotic agent (e.g., with which the RecA is administered in combination).

Growth: The term “growth”, as used herein, refers to an increase in microbial biomass. “Proliferation” (see below) refers to an increase in microbial number. Since bacterial proliferation, rather than mere increase in cell mass without cell division, is usually of primary concern, and since under most circumstances of interest herein proliferation is accompanied by an increase in microbial biomass, the term “growth” is generally understood to mean “proliferation”, and the two terms are used interchangeably herein although it is recognized that different assays may measure either or both of these parameters. For example, optical density reflects biomass and does not specifically reflect cell number, whereas an assay based on detecting colonies formed from individual cells reflects cell number rather than biomass.

High throughput screening. “High throughput screening” as used herein refers to an assay that allows for multiple candidate agents or samples to be screened substantially simultaneously. Such assays typically entail the use of microtiter (microwell) plates (e.g., plates having 24, 48, 96, 384, or 1596 wells) which are particularly convenient because a large number of assays can be carried out simultaneously, using small amounts of reagents and samples. Such assays may also advantageously minimize the number of steps such as washing cells, removing culture medium, and/or pipetting reagents.

In combination: The phrase “in combination”, as used herein, means with respect to administration of first and second agents, administration performed such that (i) a dose of the second agent is administered before more than 90% of the most recently administered dose of the first agent has been metabolized to an inactive form or excreted from the body; or (ii) doses of the first and second agents are administered within 1, 6, 12, 24, or 48 hours of each other, or (iii) the agents are administered during overlapping time periods (e.g., by continuous or intermittent infusion); or (iv) any combination of the foregoing. The agents may, but need not be, administered together as components of a single composition. The agents may be administered individually at substantially the same time (by which is meant within less than 10 minutes of one another). The agents may be administered individually within a short time of one another (by which is meant less than 3 hours, sometimes less than 1 hour, apart). The agents may, but need not, be administered by the same route of administration. When administered in combination with a second agent, the effective concentration of a first agent needed to elicit a particular biological response may be less or more than the effective concentration of the first agent when administered in the absence of the second agent, thereby allowing an adjustment of the amount dose of the first agent relative to the amount that would be needed if the first agent were administered in the absence of the second agent. In some embodiments of the invention, a lower amount of first agent (e.g., antibiotic agent) is required in the presence of the second agent (e.g., RecA inhibitor). The effects of multiple agents may, but need not be, additive or synergistic. One or more of the agents may be administered multiple times.

Infection: The term “infection”, as used herein, refers to the invasion of a host, whether the host is a vertebrate, invertebrate, fish, plant, bird, or mammal, by pathogenic microbes, e.g., bacteria, fungi, and protists. The term encompasses excessive growth of microbes that are normally present in or on the body of a mammal or other organism. More generally, a microbial infection can be any situation in which the presence of a microbial population(s) is damaging to a host organism. Thus, an organism is “suffering” from a microbial infection when excessive numbers of a microbial population are present in or on the organism's body, or when the effects of the presence of a microbial population(s) is damaging the cells or other tissue of an organism. The agents and compositions of certain embodiments of the invention are also useful in treating microbial growth or contamination of cell cultures or other media, or inanimate surfaces or objects, and nothing herein should limit the invention to treatment of higher organisms, except when explicitly so specified in the claims.

Isolated: As used herein, agent or entity is “isolated” if it is separated from at least some materials or components with which it is associated in nature or when initially generated. In general, such separation involves activity of the hand of man.

Microbial infection. The term “microbial infection” refers to the invasion of the host organism, whether the organism is a vertebrate, invertebrate, fish, plant, bird, or mammal, by pathogenic microbes, e.g., bacteria, fungi, and protists. This includes the excessive growth of microbes that are normally present in or on the body of a mammal or other organism. More generally, a microbial infection can be any situation in which the presence of a microbial population(s) is damaging to a host organism. Thus, an organism is “suffering” from a microbial infection when excessive numbers of a microbial population are present in or on the organism's body, or when the effects of the presence of a microbial population(s) is damaging the cells or other tissue of an organism. The agents and compositions of certain embodiments of the invention are also useful in treating microbial growth or contamination of cell cultures or other media, or inanimate surfaces or objects, and nothing herein should limit the invention to treatment of higher organisms, except when explicitly so specified in the claims.

Minimal inhibitory concentration (MIC): The term “minimal inhibitory concentration” (MIC) are used herein consistently with its use in the art, i.e., to indicate the concentration of an agent that will inhibit bacterial proliferation (growth) (MIC). MIC values may be for example, the concentration of agent that inhibits visible growth or may be expressed as MIC₅₀, MIC₉₀ or MIC₉₉ values i.e., the concentration of an agent that reduces bacterial proliferation to 50% or less, 10% or less, or 1% or less, respectively, of the control value that would occur in the absence of the agent. As is well known in the art, MIC can be measured by a variety of methods, including automated and non-automated methods. Suitable methods are described in publications of the Clinical Laboratory Standards Institute (CLSI), formerly the National Committee for Clinical Laboratory Standards (NCCLS), as set forth in NCCLS: Performance Standards documents.

Pharmaceutically acceptable derivative: According to the present invention, a pharmaceutically acceptable derivative of a particular chemical compound includes, but is not limited to, pharmaceutically acceptable salts, esters, salts of such esters, or any other adduct or derivative which upon administration to a patient in need thereof is capable of providing, directly or indirectly, a compound as otherwise described herein, or a metabolite or residue thereof. Thus, pharmaceutically acceptable derivatives can include salts, prodrugs, and/or metabolites of relevant compounds. The phrase “pharmaceutically acceptable derivative” may also encompass quaternization of any basic nitrogen-containing groups of the compounds disclosed herein. Water or oil-soluble or dispersable products may be obtained by such quaternization.

Pharmaceutically acceptable salt: As used herein, the term “pharmaceutically acceptable salt” refers to those salts which are, within the scope of sound medical judgment, suitable for use in contact with the tissues of humans and lower animals without undue toxicity, irritation, allergic response and the like, and which are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorally active metabolite or residue thereof. As used herein, the term “inhibitorally active metabolite or residue thereof” means that a metabolite or residue thereof acts as a RecA inhibitor.

A wide variety of appropriate pharmaceutically acceptable salts are well known in the art. For example, S. M. Berge et al., describe pharmaceutically acceptable salts in detail in J. Pharmaceutical Sciences, 66:1, 1977, incorporated herein by reference. Pharmaceutically acceptable salts of the compounds of this invention include those derived from suitable inorganic and organic acids and bases.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Examples of pharmaceutically acceptable salts derived from appropriate bases include alkali metal, alkaline earth metal, ammonium and N⁺(C₁₋₄ alkyl)₄ salts.

Representative pharmaceutically acceptable alkali or alkaline earth metal salts include sodium, lithium, potassium, calcium, magnesium, and the like.

Further pharmaceutically acceptable salts include, when appropriate, nontoxic ammonium, quaternary ammonium, and amine cations, for example formed using counterions such as halide, hydroxide, carboxylate, sulfate, phosphate, nitrate, lower alkyl sulfonate and aryl sulfonate.

Pharmacophore: As used herein, the term “pharmacophore” refers to a collection of chemical features, three-dimensional constraints that represent specific characteristics responsible for a compound's activity. The pharmacophore includes surface-accessible features, hydrogen bond donors and acceptors, charged/ionizable groups, and/or hydrophobic patches, among other features.

Physiologically acceptable carrier or excipient: As used herein, the term “physiologically acceptable carrier or excipient” refers to a carrier medium or excipient which does not interfere with the effectiveness of the biological activity of the active ingredients and which is not excessively toxic to the host at the concentrations at which it is administered. The term includes solvents, dispersion media, coatings, antibacterial and antifungal agents, isotonic agents, absorption delaying agents, and the like. The use of such media and agents for the formulation of pharmaceutically active substances is well-known in the art (see, for example, “Remington's Pharmaceutical Sciences”, E. W. Martin, 18^{th} Ed., 1990, Mack Publishing Co.: Easton, Pa., which is incorporated herein by reference in its entirety).

Potentiate: The term “potentiate”, as used herein, means to enhance or increase at least one biological effect or activity of a biologically and/or pharmacologically active agent so that either (i) a given concentration or amount of the agent results in a greater biological effect or activity when the agent is potentiated than the biological effect or activity that would result from the same concentration or amount of the agent when not potentiated; or (ii) a lower concentration or amount of the agent is required to achieve a particular biological effect or activity when the agent is potentiated than when the agent is not potentiated; or (iii) both (i) and (ii). The biological effect or activity may be, for example, the ability to catalyze or inhibit one or more chemical reactions, the ability to activate or inhibit a biological or biochemical pathway, the ability to reduce or inhibit microbial proliferation, the ability to kill a microorganism, etc. An agent whose presence potentiates another agent may be referred to as a “potentiating agent”. A potentiating agent may show biological activity by itself, or may exhibit biological activity only when used in combination with a biologically and/or pharmacologically active agent.

Proliferation: The term “proliferation” refers to an increase in microbial number. Since bacterial proliferation, rather than mere increase in cell mass without cell division, is usually of primary concern, and since under most circumstances of interest herein proliferation is accompanied by an increase in microbial biomass, the term “growth” is generally understood to mean “proliferation”, and the two terms are used interchangeably herein although it is recognized that different assays may measure either or both of these parameters. For example, optical density reflects biomass and does not specifically reflect cell number, whereas an assay based on detecting colonies formed from individual cells reflects cell number rather than biomass.

Protist. The term “protist” refers to any member of a diverse group of organisms, comprising those eukaryotes that are not animals, plants or fungi. Protists can be unicellular or multicellular. Protists are group in three subcategories: animal-like protists, fungus-like protists, and plant-like protists.

Purified: As used herein, the term “purified” refers to agents or entities that have been separated from most of the components with which they are associated in nature or when originally generated. In general, such purification involves action of the hand of man. Purified agents or entities may be partially purified, substantially purified, or pure. Such agents or entities may be, for example, 50%, 60%, 70%, 75%, 80%, 85%, 90%, 95%, 96%, 97%, 98%, 99%, or more than 99% pure.

RecA Inhibitor: According to the present invention, an agent is a RecA inhibitor if one or more RecA activities is reduced in the agent's presence as compared with its absence, or if the level or amount of RecA protein or gene product is reduced in the agent's presence as compared with its absence. In certain embodiments, RecA inhibitors act directly on RecA in that they physically interact with RecA. In other embodiments, inhibitors act indirectly on RecA.

Small Molecule: In general, a small molecule is understood in the art to be an organic molecule that is less than about kilodaltons (KDa) in size. In some embodiments, the small molecule is less than about 3 KDa, 2 KDa, or 1 KDa. In some embodiments, the small molecule is less than about 800 daltons (Da), 600 Da, 500 Da, 400 Da, 300 Da, 200 Da, or 100 Da. In some embodiments, small molecules are non-polymeric. In some embodiments, small molecules are not amino acids. In some embodiments, small molecules are not nucleotides. In some embodiments, small molecules are not saccharides.

Subject: A “subject”, as used herein, is an individual to whom an agent is to be delivered, e.g., for experimental, diagnostic, and/or therapeutic purposes. Subjects of interest herein include animals, particularly agriculturally significant animals or companion animals (e.g., cows, sheep, goats, horses, swine, dogs, cats, rabbits, birds, poultry, fish, etc.), laboratory animals (e.g., mice, rats) primates, or humans.

Sublethal: A “sublethal” concentration of an antibiotic refers to a concentration that is less than the MIC of the antibiotic. In certain embodiments of the invention a sublethal concentration is not sufficient to significantly reduce the growth rate (proliferation) of a microbial cell, e.g., the growth rate is reduced by less than 20%, preferably less than 10%. Such a concentration is referred to herein as a “non-inhibiting concentration”. A “lethal” concentration of an antibiotic is one that is equal to or greater than the MIC and would ultimately result in microbial death and complete or essentially complete sterilization of a culture medium containing the microbe if continued indefinitely assuming that no resistant strains arise during the incubation period.

Sensitive/susceptible and resistant. A microorganism is “sensitive” or “susceptible” to an agent if the agent inhibits proliferation of the microorganism and/or kills the microorganism when contacted with the agent at a particular concentration. Sensitivity may be assessed using any of a variety of methods known in the art. A microorganism that is not “sensitive” is considered “resistant”, i.e., the microorganism can survive and proliferate in the presence of the agent. Methods for assessing sensitivity typically involve determining the MIC by methods such as the broth microdilution method, agar dilution, and the agar disk diffusion method. The MIC may then be compared with a predefined “breakpoint”, wherein a MIC greater than the breakpoint indicates that the microorganism is resistant to the agent and a MIC equal to or below the breakpoint indicates that the microorganism is sensitive to the agent. Sensitivity and/or resistance may be assessed according to the guidelines and methods established by the Clinical Laboratory Standards Institute (CLSI), formerly the National Committee for Clinical Laboratory Standards (NCCLS), as set forth in NCCLS: Performance Standards for Antimicrobial Susceptibility Testing; Fourteenth Informational Supplement. NCCLS document M100-S14. Wayne, Pa.: NCCLS 2004; NCCLS: Performance Standards for Antimicrobial Disk Susceptibility Tests; Approved Standard-Eighth Edition. NCCLS document M2-A8. Wayne, Pa.: NCCLS 2003; or NCCLS: Methods for Dilution Antimicrobial Susceptibility Tests for Bacteria that Grow Aerobically: Approved Standard-Sixth Edition. NCCLS document M7-A6. Wayne, Pa.: NCCLS 2003. “Intrinsic resistance” means that a bacterial species is inherently resistant to the effects of an antibacterial agent. “Acquired resistance” means that a bacterial species, subtype, or strain has acquired a mechanism of resistance since the introduction of the antibacterial agent into use. Resistance may, for example, be acquired by mutation of a target gene, by overexpression of an efflux pump, etc. A subpopulation of a bacterial species that has acquired resistance may be considered a distinct strain of that species.

Survival: The term “survival”, as used herein, refers to an ability of microbial cells to grow in the presence of one or more antibiotic agent(s) present above the relevant minimum inhibitory concentration. In some embodiments, survival is assessed at a concentration that is at or above a multiple of MIC (e.g., 2×, 4×, 5×, 6×, 8×, 10×, etc).

Toxicity. “Toxicity” refers to any adverse and/or undesired effect of a composition on the metabolism or functioning of a cell, tissue, organ or body part, or subject. The amount of toxicity associated with a composition may vary with several conditions including, but not limited to, the amount of composition present, the components present in the composition, the formulation of the composition, the environmental conditions and physiological state of the cell, tissue, organ or body part, or subject, etc.

Treatment: As used herein, the term “treatment” refers to the provision of any type of medical or surgical management to a subject. Treatment can include, but is not limited to, administering a pharmaceutical composition to a subject. Treatment is typically undertaken in an effort to alter the course of a disease, disorder, or undesirable condition in a manner beneficial to the subject. The effect of treatment can generally include reversing, alleviating, reducing severity of, delaying the onset of, inhibiting the progression of, and/or reducing the likelihood of occurrence or reoccurrence of the disease, disorder, or condition to which such term applies, or one or more symptoms or manifestations of such disease, disorder or condition. A composition of this invention can be administered to a subject who has developed an infection or is at increased risk of developing an infection relative to a member of the general population. A composition of this invention can be administered prophylactically, i.e., before development of any symptom or manifestation of a condition. Typically in this case the subject will be at risk of developing the condition. For example, an inventive composition can be administered prior to exposure of the subject to an infectious agent or prior to the occurrence of a pathogenic event.

Unit Dosage Form: A “unit dosage form”, as that term is used herein, refers to physically discrete units suited as unitary dosages for the subject to be treated; each unit containing a predetermined quantity of active agents) calculated to produce the desired therapeutic effect in association with the required pharmaceutical carrier

### DETAILED DESCRIPTION OF CERTAIN EMBODIMENTS

The present invention provides RecA inhibitors, compositions containing them, systems for identifying or characterizing them, and methods of using them. In some embodiments, RecA inhibitors are identified by RecA luciferase assays. In some embodiments, RecA inhibitors can be used to potentiate the activity of antibiotic agents. When RecA inhibitors are administered in combination with one or more such antibiotic agents whose activity they potentiate, the antibiotic agent(s) may often be utilized at a lower dose, and/or less frequent dosing regimen than their conventional dose and/or schedule. In some embodiments, addition of one or more RecA inhibitors to an antibiotic therapy regimen significantly reduces the survival of bacteria at conventional therapeutic antibiotic agent dosing. In some embodiments, inventive RecA inhibitors reduce the incidence of resistance developed toward one or more antibiotic agents. In some embodiments, RecA inhibitors retard resistance developed toward one or more antibiotic agents. In some embodiments, RecA inhibitors can be used as stand-along anti-infective compounds to treat microbial infections.

Each of these embodiments, and others, is discussed in more detail below.

### I—Rec A

The RecA protein is an important sensor and activator in response to DNA damage and plays a major role in inducing the SOS response pathway following such damage. It is known that RecA is also involved in other cellular processes in addition to recombination and DNA damage repair.

One fundamental event in both homologous recombination and SOS response induction is the formation of a RecA-ssDNA-ATP nucleoprotein filament. In this conformation, RecA acts as both a recombinase and co-protease. In the latter function, it activates the SOS response by cleaving the LexA repressor protein, which results in the induction of genes that are repressed by LexA under normal conditions. Over 30 SOS genes, and UmuD, a sub-unit of polymerase IV, also involved in the SOS response, are induced (Courcelle and Hanawalt, Annu Rev Genet., 2003, 37: 611-646; Sutton et al., Annu Rev Genet. 2000, 34: 479).

Another fundamental role of RecA is to maintain the integrity of the genetic material. The binding of RecA to single-stranded DNA regions that block replication forks serves as the sensor that replication is blocked and maintains the integrity of the replication fork itself until replication can resume (Courcelle and Hanawalt, Annu Rev Genet., 2003, 37: 611-646).

The RecA protein is highly conserved and is cross species functional. For example, recA homologs from Yersinia pestis, Bacillus anthracis and M. tuberculosis have been shown to complement the E. coli recA-mutation (Suchkov and Mishan'kin, Mol Gen Mikrobiol Virusol., 1989 5: 34; Ko et al., J Bacteriol., 2002, 184: 3917; Nair and Steyn, J Gen Microbiol., 1991, 137: 2409). Thus, at least some inhibitors of RecA identified using the RecA protein from one species would be expected to show activity in a wide variety of bacteria and other microorganisms.

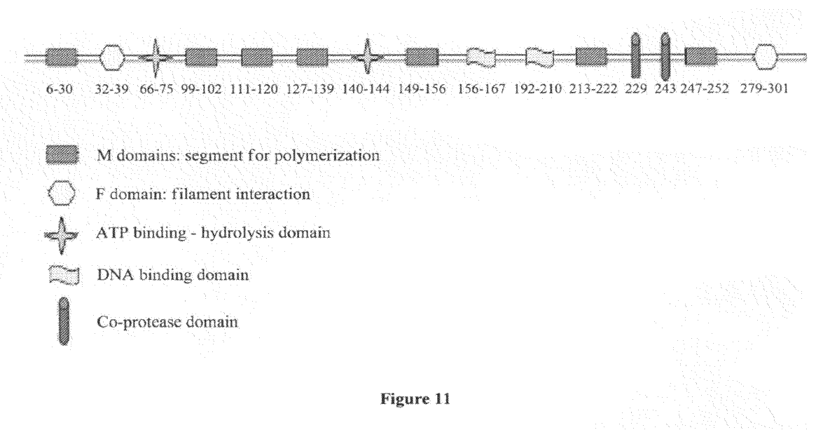

The RecA protein has many functional features that present points of intervention to inhibit its activity in accordance with the present invention. Multiple alignment of the sequence reveals a canonical structure of RecA-like proteins consisting of distinct segments or motifs (FIG. 11). These segments or modules are highly conserved and have been assigned functional roles based on genetic, biochemical and structural studies. Such modules are involved in DNA damage recognition and binding, monomer interaction, filament formation, helicase motifs, ATP binding and hydrolysis, recombination, replication and co-protease activity. Mutational studies have identified residues that are critical to these processes.

For example, the Gly157 change generates a constitutive co-protease form of RecA and results in a lower survival in response to UV treatment, a phenotype itself associated with a low recombination competent form of RecA. This information has been used to precisely map regions of RecA to be targeted for compound discovery, e.g., using computational approaches.

For example, RecX (also called OraA) is an inhibitor of RecA for both recombinase and co-protease activities (Stohl et al., J. Biol. Chem., 2003, 278: 2278,). RecX appears to inhibit the ATPase activity of RecA. Genetic and biochemical evidence identifies sites of interaction between RecA and LexA, suggesting that amino acids at positions 67, 154-157, 229 and 243 are responsible at least in part for the binding to LexA (VanLook et al., 2003, J Mol. Biol., 333: 345). Also, amino acid changes at positions 122-123 and 150-161 dramatically decrease the ability of mutant cells to survive in response to UV radiation treatment. A domain in RecA that likely forms part of the co-protease substrate binding site has also been identified (Nastri et al., Mol. Microbiol., 1997, 25: 967).

The present Applicant has discovered that RecA inhibition combined with bactericidal antibiotics can be used to overcome antimicrobial resistance. For example, the Applicant has demonstrated that inactivation of RecA resulted in three orders of magnitude greater cell killing by, e.g., Norfloxacin, Ciprofloxain and Gentamicin. RecA⁻ strains were also significantly sensitized to Ampicillin. Additionally, Norfloxacin resistance in E. coli due to gyrA mutations could be overcome by inactivation of RecA. Furthermore, recA⁻ S. aureus exhibited attenuated virulence in three animal models. See, International Application No. PCT/US07/03712, filed on Feb. 13, 2007, entitled “RecA Inhibitors with Antibiotic Activity, Compositions and Methods of Use,” International Application No. PCT/US07/03698, filed on Feb. 13, 2007, entitled “Compositions and Methods for Antibiotic Potentiation and Drug Discovery,” and International Application No. PCT/US07/075,093, filed on Aug. 2, 2007, entitled “Compositions and Methods for Potentiating Antibiotic Activity,” the contents of all of which are hereby incorporated by reference.

II—Identification of Rec A Inhibitors Using RecA-luciferase assays

Among other things, the present invention provides high throughput in vitro RecA-luciferase assays to identify RecA inhibitors (see Example 1). RecA is a DNA-dependent ATPase (i.e., it catalyzes the reaction adenosine triphosphate [ATP]→adenosine diphosphate [ADP]). An assay was developed for RecA ATPase activity based on detection of the amount of ATP remaining in a reaction mixture following incubation of RecA protein, DNA, and ATP. In an exemplary assay, E. coli RecA protein is incubated in reaction buffer with DNA, ATP, and any test compound or compounds for a measured amount of time. The quantity of ATP remaining after the RecA reaction can be quantitated using a subsequent luciferase assay. The luciferase emits more light as more ATP is available. In some embodiments, the amount of ATP remaining can be compared to controls containing either a known inhibitor (full inhibition control) or no test compound (full activity control). In some embodiments, a positive control (having no DNA added to the well) and negative control (having DNA and no compounds) are run with each screen. Positive responses are based upon the percent reduction of activity of luciferase, with a high percentage representing increased RecA inhibition. Percent inhibition can be calculated according to the following equation:

$$\frac{( RLU in presence of compound ) - ( RLU in DMSO )}{( RLU in absence of DNA ) - ( RLU in DMSO )} .$$

Various candidate agents can be screened for potential RecA inhibitors in accordance with the present invention. Exemplary candidate agents include, but are not limited to, chemical compounds, small molecules, proteins or peptides, antibodies, co-crystals, nano-crystals, microorganisms (e.g., virus, bacteria, fungi, etc.), nucleic acids (e.g., DNAs, RNAs, DNA/RNA hybrids, siRNAs, shRNAs, miRNAs, ribozymes, aptamers, etc.), carbohydrates (e.g. mono-, di-, or poly-saccharides), lipids (e.g., phospholipids, triglycerides, steroids, etc.), natural products, any combination thereof. Candidate agents can also be designed using computer-based rational drug design methods. For example, portions of RecA can be selected and used to design small molecules that can potentially bind to RecA and inhibit one or more activities of RecA (e.g., DNA binding, monomer interaction, helicase activity, filament formation, ATP binding and/or hydrolysis, co-protease activity (e.g., toward LexA and/or UmuD), recombinase activity, replication function, and combinations thereof) using computational methods.

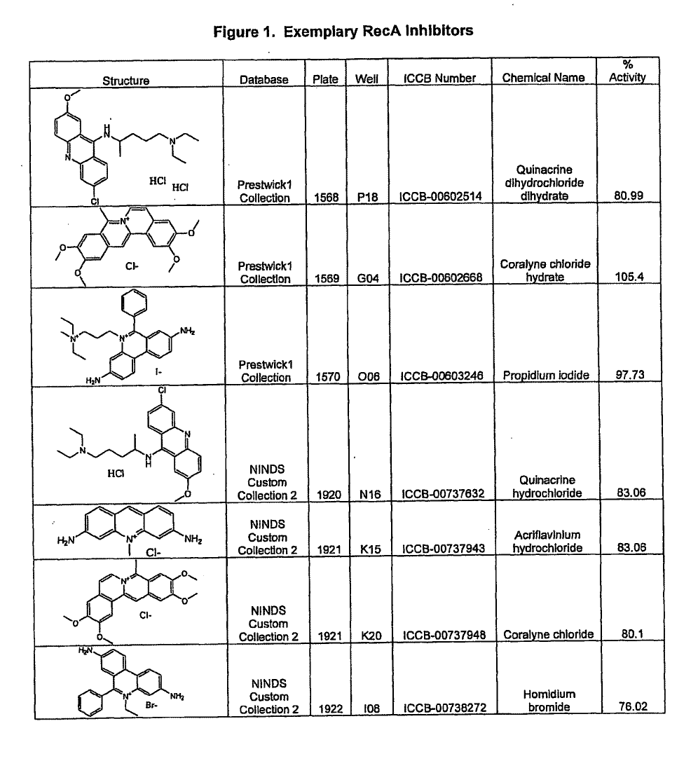

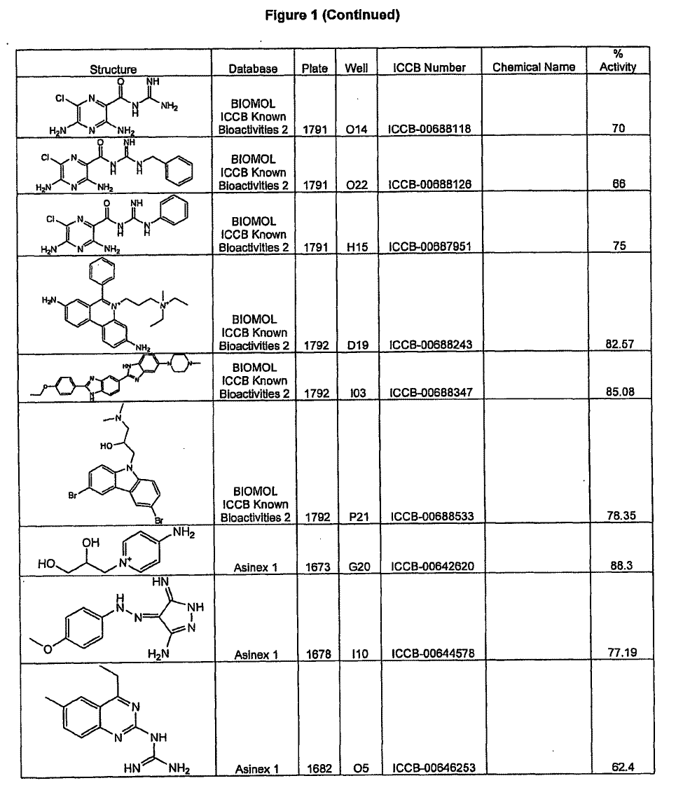

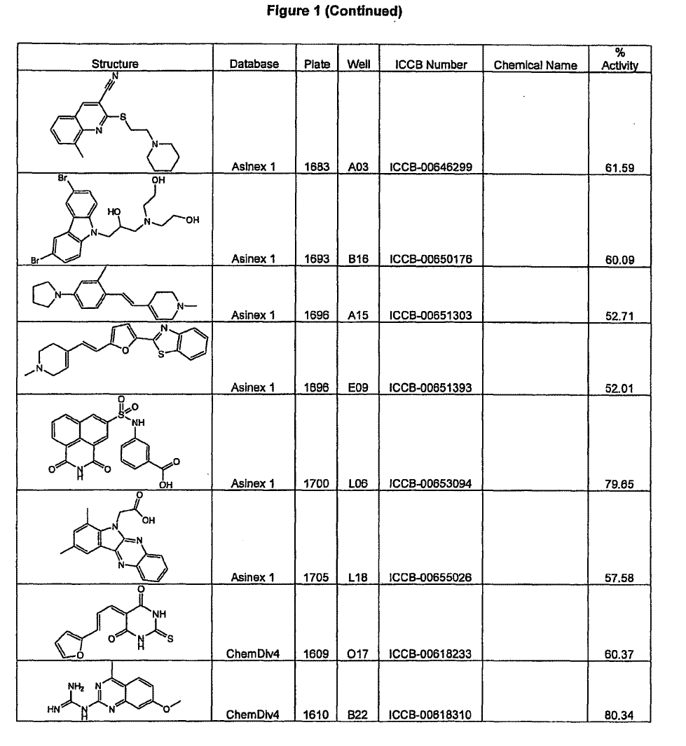

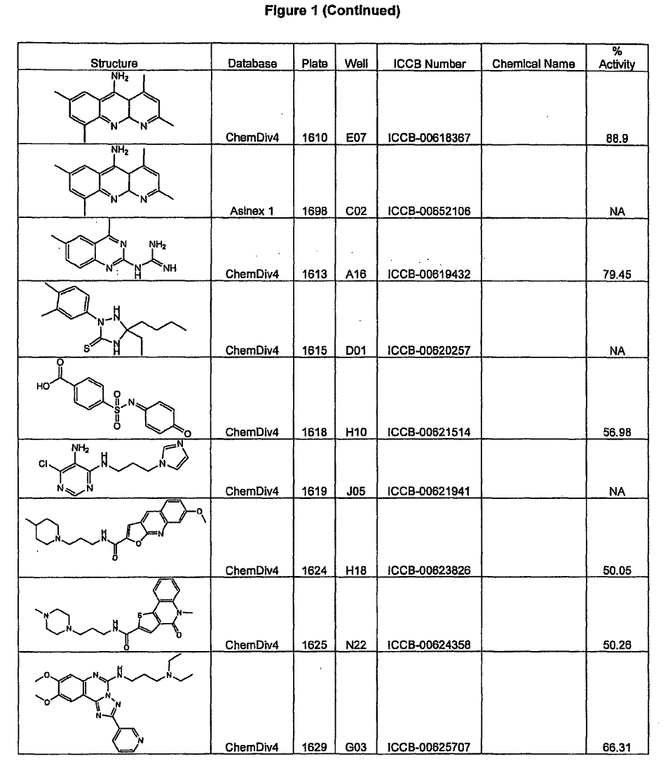

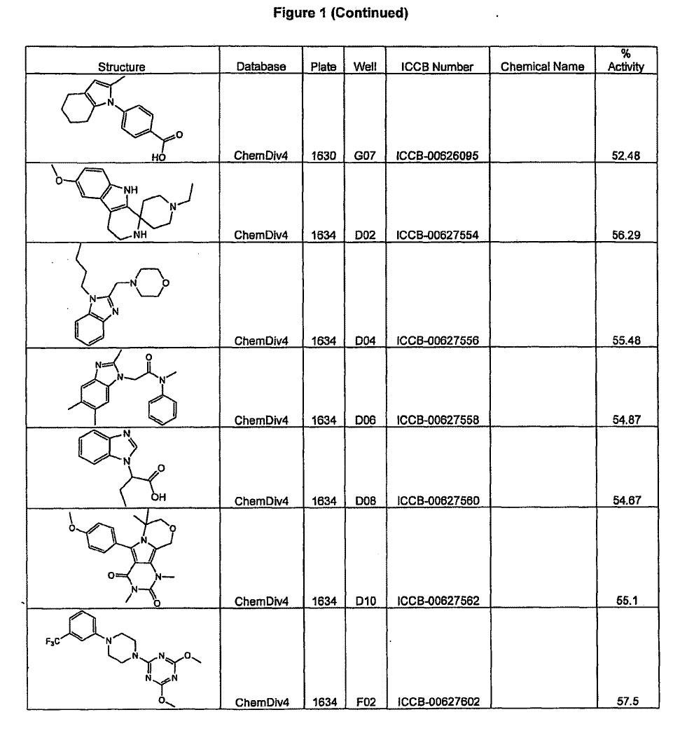

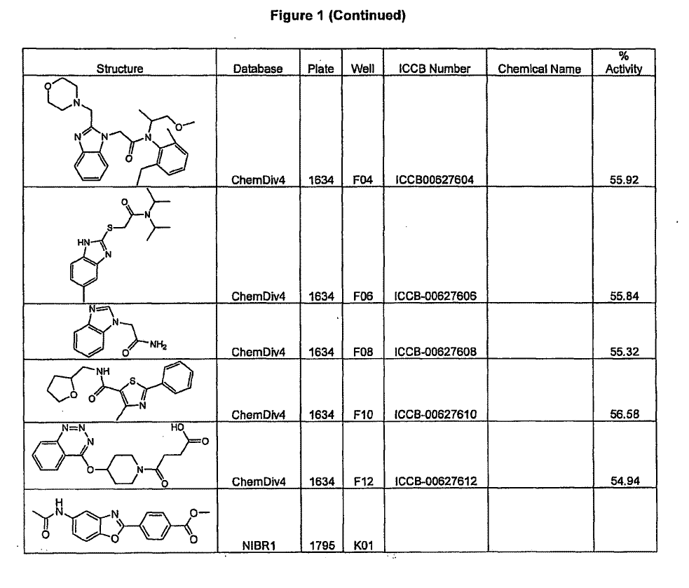

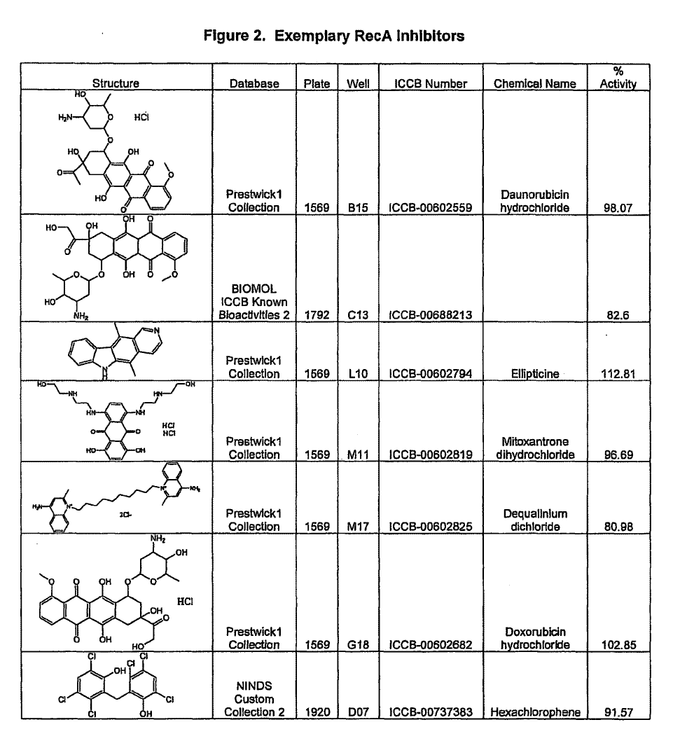

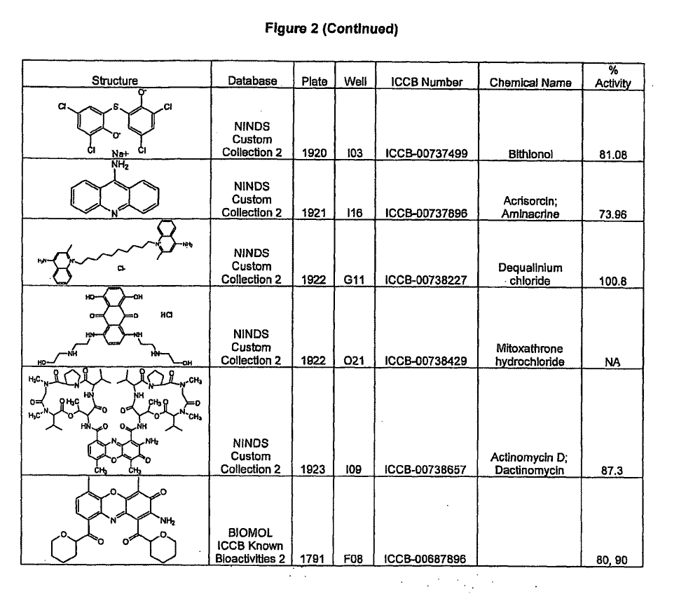

In some embodiments, public libraries containing drugs (including FDA approved drugs) can be screened to identify existing compounds whose RecA inhibitory function are previously unknown. In some embodiments, modified libraries containing derivatives or analogues of existing compounds can be synthesized using methods well known in the art and screened to identify novel or improved RecA inhibitors. In some embodiments, compound libraries synthesized de novo can be screened to identify novel compounds that have RecA inhibitory functions. As described in the Examples section below, the present Applicant has screened over 22,000 compounds. Exemplary RecA inhibitors identified in accordance with the present invention are presented in FIGS. 1 and 2.

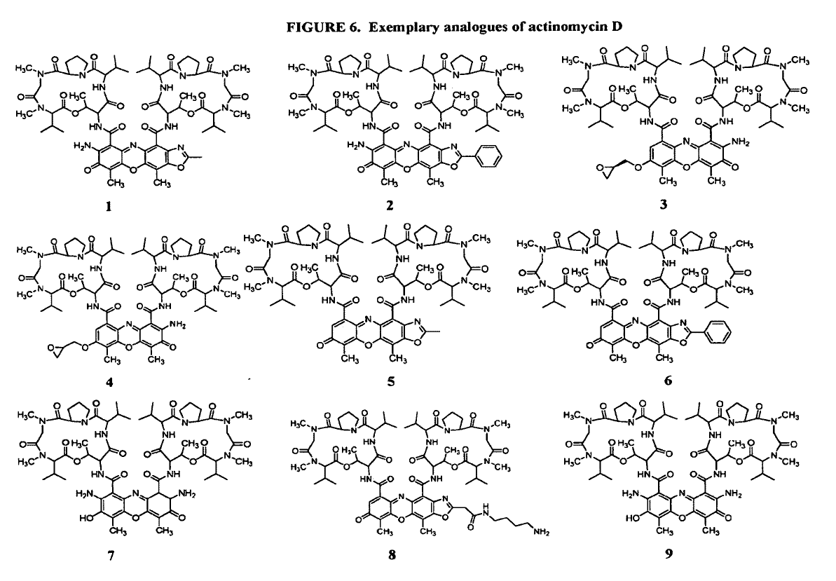

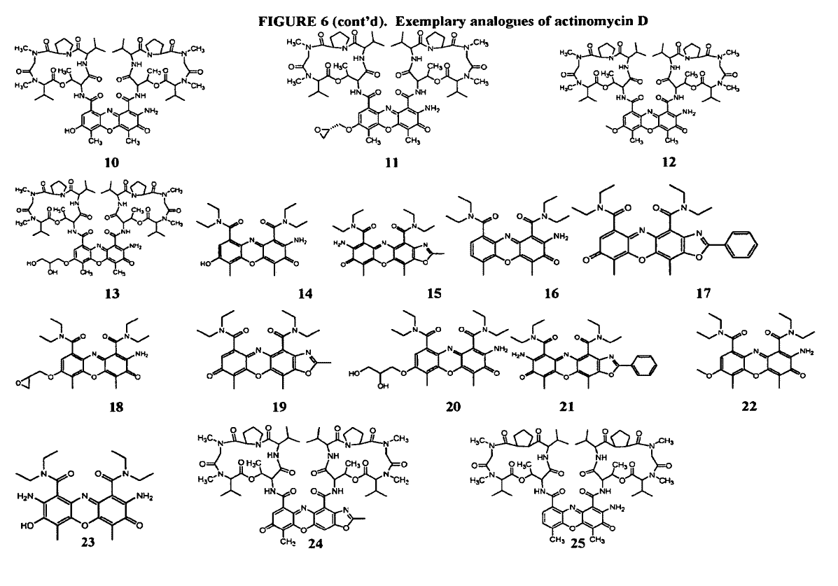

In some embodiments, RecA inhibitors are small molecule agents, typically having a cyclic moiety (e.g., including one or more aryl rings). Certain RecA inhibitors according to the present invention are flavones; certain RecA inhibitors according to the present invention are bisflavones. In some embodiments of the invention, the RecA inhibitor is or includes actinomycin D or a derivative or an analogue thereof (see FIG. 6). In some embodiments, RecA inhibitors have a polycyclic core.

Alternatively or additionally, compounds identified in the luciferase assay can be tested for pharmacological or biological activity using additional in vitro assays (e.g., using cell survival assays or cell growth assays to test antibiotic-potentiating activity) or in animal models for their ability to treat a disease, condition, or syndrome of interest (e.g., in vivo anti-microbial assays using immunocompromised mice). Compounds displaying desired pharmacological or biological activity can be considered as lead compounds.

Molecular Modeling

Compounds identified herein (e.g., lead compounds) can be used in the design of derivatives or analogues possessing useful pharmacological activity and/or physiological profiles. For example, following the identification of a lead compound, molecular modeling techniques can be employed, which have proven to be useful in conjunction with synthetic efforts, to design variants of the lead compound that can be more effective (e.g., more potent inhibition of RecA, increased antimicrobial activity, more efficient potentiation of other antibiotics, or better pharmacological properties). Exemplary molecular modeling techniques may include, but are not limited to, Pharmacophore Modeling (cf Lamothe, et al. 1997, J. Med. Chem. 40: 3542; Mottola et al. 1996, J. Med. Chem. 39: 285; Beusen et al. 1995, Biopolymers 36: 181; P. Fossa et al. 1998, Comput. Aided Mol. Des. 12: 361), QSAR development (Siddiqui et al. 1999, J. Med. Chem. 42: 4122; Barreca et al. 1999 Bioorg. Med. Chem. 7: 2283; Kroemer et al. 1995, J. Med. Chem. 18: 4917; Schaal et al. 2001, J. Med. Chem. 44: 155; Buolamwini & Assefa 2002, J. Mol. Chem. 45: 84), and Virtual docking and screening/scoring (cf Anzini et al. 2001, J. Med. Chem. 44: 1134; Faaland et al. 2000, Biochem. Cell. Biol. 78: 415; Silvestri et al. 2000, Bioorg. Med. Chem. 8: 2305; J. Lee et al. 2001, Bioorg. Med. Chem. 9: 19). Further examples of the application of such techniques can be found in several review articles, such as Rotivinen et al., 1988, Acta Pharmaceutical Fennica 97:159-166; Ripka, 1998, New Scientist 54-57; McKinaly & Rossmann, 1989, Annu. Rev. Pharmacol. Toxiciol. 29:111-122; Perry & Davies, QSAR: Quantitative Structure-Activity Relationships in Drug Design pp. 189-193 (Alan R. Liss, Inc. 1989); Lewis & Dean, 1989, Proc. R. Soc. Lond. 236:125-140 and 141-162; Askew et al., 1989, J. Am. Chem. Soc. 111: 1082-1090.

Molecular modeling tools employed may include those from Tripos, Inc., St. Louis, Mo. (e.g., Syby/UNITY, CONCORD, DiverseSolutions), Accelerys, San Diego, Calif. (e.g., Catalyst, Wis. Package {BLAST, etc.}), Schrodinger, Portland, Oreg. (e.g., QikProp QikFit, Jaguar) or other such vendors as BioDesign, Inc. (Pasadena, Calif.), Allelix, Inc. (Mississauga, Ontario, Canada), and Hypercube, Inc. (Cambridge, Ontario, Canada), and may include privately designed and/or “academic” software (e.g. RNAMotif, MFOLD). These application suites and programs include tools for the atomistic construction and analysis of structural models for drug-like molecules, proteins, and DNA or RNA and their potential interactions. They also provide for the calculation of important physical properties, such as solubility estimates, permeability metrics, and empirical measures of molecular “druggability” (e.g., Lipinski “Rule of 5” as described by Lipinski et al. 1997, Adv. Drug Delivery Rev. 23: 3-25). Most importantly, they provide appropriate metrics and statistical modeling power (such as the patented CoMFA technology in Sybyl as described in U.S. Pat. Nos. 6,240,374 and 6,185,506) to develop Quantitative Structural Activity Relationships (QSARs) which are used to guide the synthesis of more efficacious clinical development candidates while improving desirable physical properties, as determined by results from the aforementioned secondary screening protocols.

Derivatives or Analogues

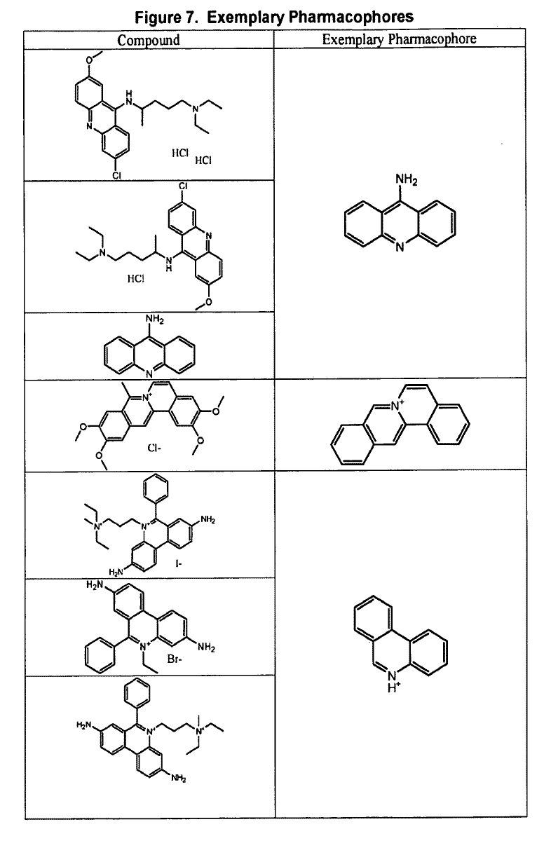

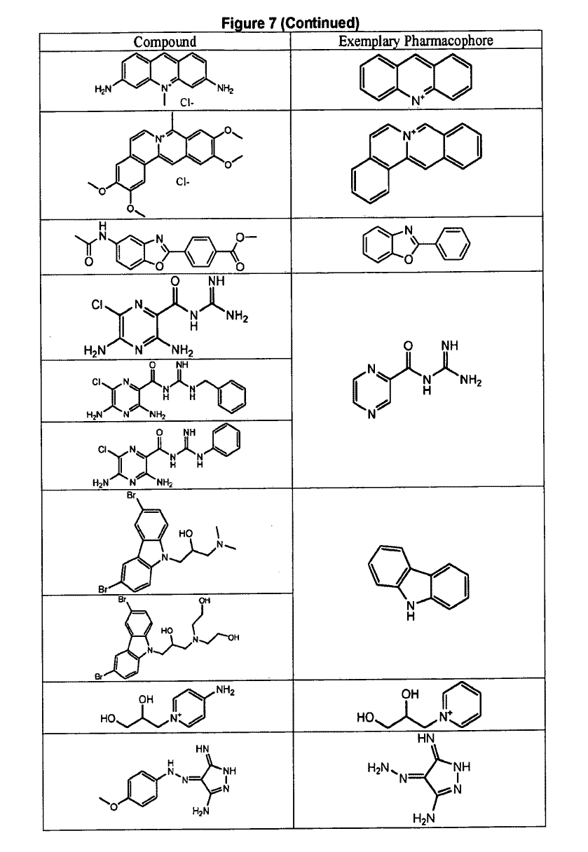

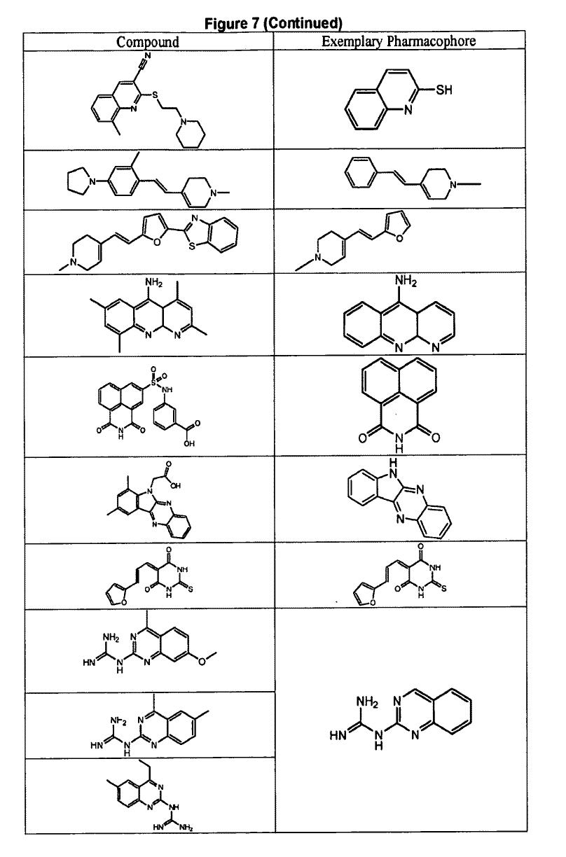

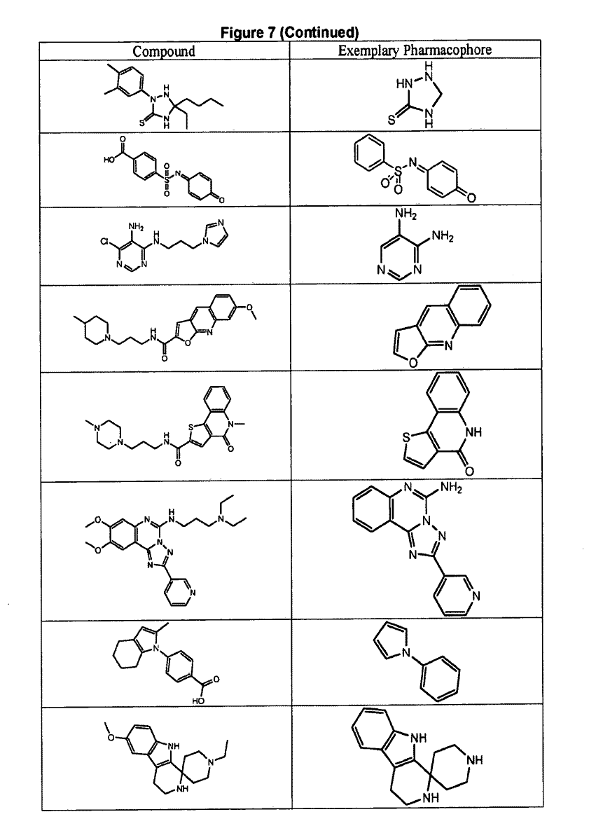

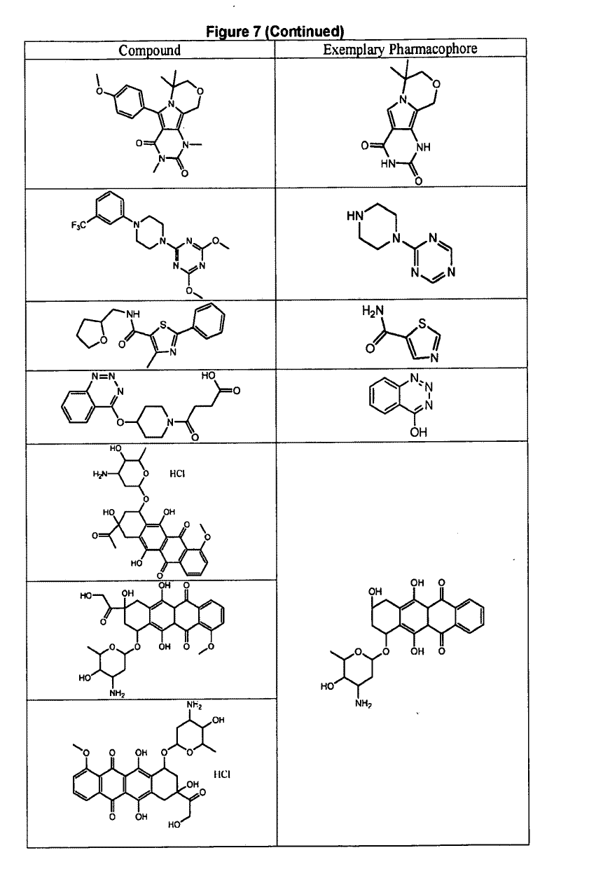

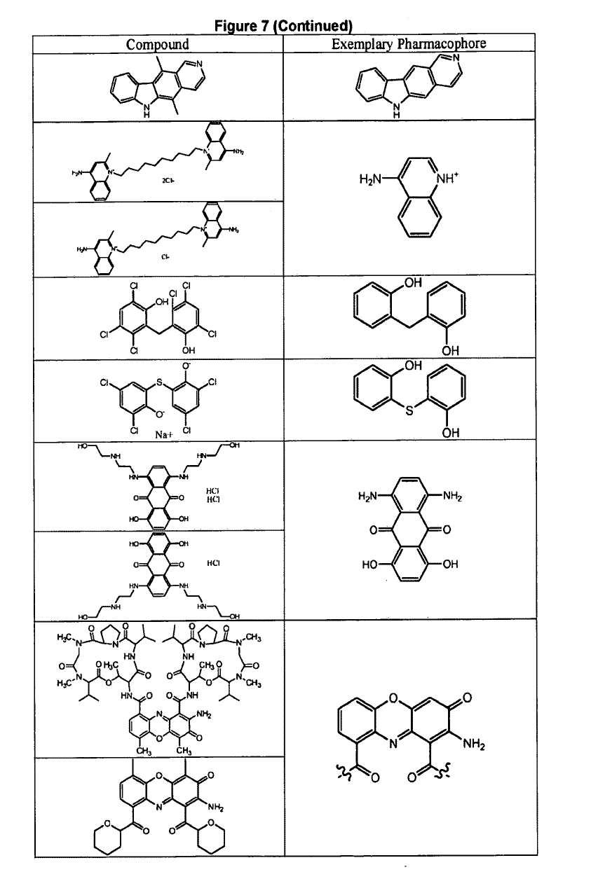

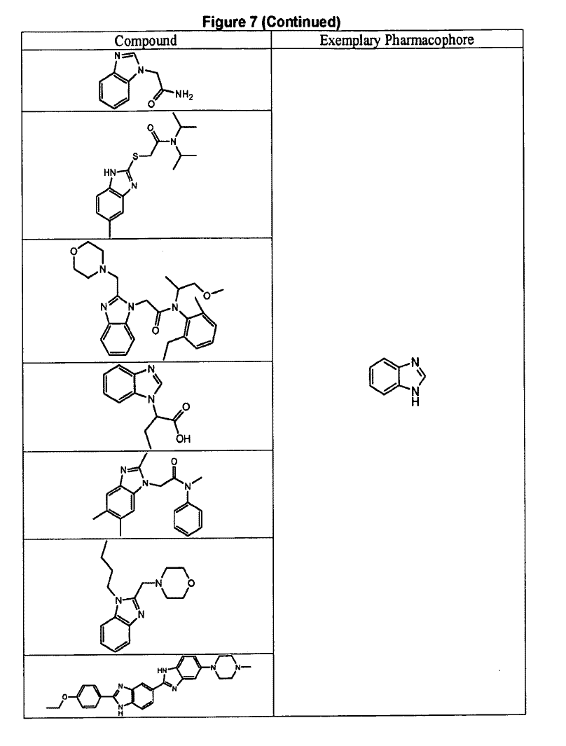

Thus, RecA inhibitors of the present invention also include analogues or derivatives of the compounds directly identified in the RecA luciferase assay. In some embodiments, RecA inhibitors of the present invention include analogues or derivatives of the compounds as shown in FIGS. 1 and 2. In some embodiments, derivatives or analogues of the compounds shown in FIGS. 1 and 2 are developed based on molecule modeling using methods described herein and known in the art. In some embodiments, derivatives or analogues of compounds shown in FIGS. 1 and 2 are developed based on pharmacophore modeling techniques (e.g., Example 6). In some embodiments, RecA inhibitors of the present invention include derivatives or analogues that have a pharmacophore selected from the pharmacophore structures shown in FIG. 7. In some embodiments, RecA inhibitors of the present invention include derivatives or analogues that have a pharmacophore selected from the pharmacophore structures shown in FIG. 7 and at least one side chain or ring linked to the pharmacophore that is present in the corresponding original compound (e.g., a functional group). In some embodiments, RecA inhibitors of the present invention include derivatives or analogues that have a pharmacophore selected from the pharmacophore structures shown in FIG. 7 and side chains or rings linked to the pharmacophore substantially similar to the side chains or rings present in the corresponding original hit compound.

As used herein, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical linkage bonds (e.g., rotatable linkage bonds). In some embodiments, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical atom coordinates defining the structures, or equivalent structures having a root mean square of deviation less than about 5.0 Å (e.g., less than about 4.5 Å, less than about 4.0 Å, less than about 3.5 Å, less than about 3.0 Å, less than about 2.5 Å, less than about 2.0 Å, less than about 1.5 Å, or less than about 1.0 Å). In some embodiments, two chemical structures are considered “substantially similar” if they share at least 50% (e.g., at least 55%, at least 60%, at least 65%, at least 70%, at least 75%, at least 80%, at least 85%, at least 90%, at least 95%, at least 98%, or at least 99%) identical atom coordinates defining surface-accessible features (e.g., hydrogen bond donors and acceptors, charged/ionizable groups, and/or hydrophobic patches), or equivalent features having a root mean square of deviation less than about 5.0 Å(e.g., less than about 4.5 Å, less than about 4.0 Å, less than about 3.5 Å, less than about 3.0 Å, less than about 2.5 Å, less than about 2.0 Å, less than about 1.5 Å, or less than about 1.0 Å). As used herein, a derivative or an analog of a compound is not the compound itself.

In some embodiments, RecA inhibitors of the present invention include derivatives or analogues of the compounds identified from the RecA luciferase assay but excluding the original compounds themselves as shown in FIGS. 1 and 2.

In certain embodiments, the present invention provides the following exemplary classes of compounds based on the compounds identified in the screen.

In certain embodiments, a compound of the present invention is represented by the formula:

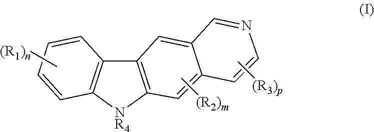

wherein

n is an integer between 0 and 4, inclusive;

m is an integer between 0 and 2, inclusive;

p is an integer between 0 and 3, inclusive;

each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —OC(O)R_{A}; —OC(O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₂ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N(R_{B})₂; —NHC(O)R_{B}; —OC(O)R_{B}; —OC(O)OR_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; —OC(O)R_{C}; —OC(O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₄ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R_{D}; —CO₂R_{D}; —N(R_{D})₂; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, R₄ is hydrogen.

In certain embodiments, the compound of formula (I) is not of formula:

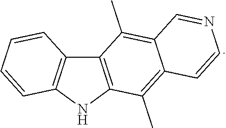

In certain embodiments, the compound is of the formula (Ia):

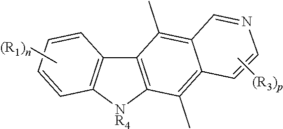

wherein n, p, R₁, R₃, and R₄ are as defined above. In certain embodiments, R₄ is hydrogen.

In certain embodiments, the compound is of the formula (Ib):

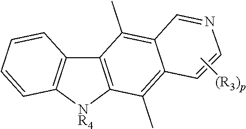

wherein p, R₃, and R₄ are as defined above. In certain embodiments, R₄ is hydrogen.

In certain embodiments, the compound is of the formula (Ic):

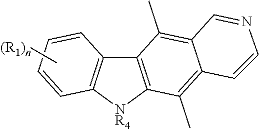

wherein n, R₁, and R₄ are as defined above. In certain embodiments, R₄ is hydrogen.

In certain embodiments, the compound is of the formula (Id):

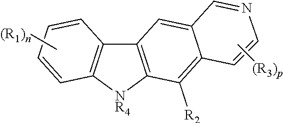

wherein n, p, R₁, R₂, R₃, and R₄ are as defined above. In certain embodiments, R₄ is hydrogen.

In certain embodiments, the compound is of the formula (Ia):

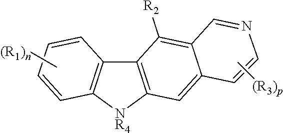

wherein n, p, R₁, R₂, R₃, and R₄ are as defined above. In certain embodiments, R₄ is hydrogen.

In certain embodiments, a compound of the present invention is represented by the formula:

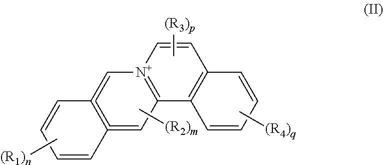

wherein

n is an integer between 0 and 4, inclusive;

m is an integer between 0 and 2, inclusive;

p is an integer between 0 and 2, inclusive;

q is an integer between 0 and 4, inclusive;

each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —OC(O)R_{A}; —OC(O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₂ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N(R_{B})₂; —NHC(O)R_{B}; —OC(O)R_{B}; —OC(O)OR_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; —OC(O)R_{C}; —OC(O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₄ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{D}; —SOR_{D}; —SO₂R_{D}; —NO₂; —N(R_{D})₂; —NHC(O)R_{D}; —OC(O)R_{D}; —OC(O)OR_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof. In certain embodiments, p is 0.

In certain embodiments, the compound of formula (II) is not of the formula:

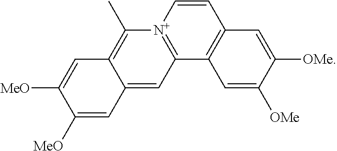

In certain embodiments, the compound of formula (II) is of the formula:

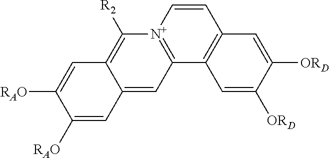

wherein R_{A}, R_{D}, and R₂ are defined above. In certain embodiments, R_{A} and R_{D} are C₁-C₆ alkyl. In certain embodiments, at least one of R_{A} and R_{D} are not methyl. In certain embodiments, R₂ is C₁-C₆ alkyl. In certain embodiments, R₂ is methyl. In certain embodiments, R₂ is not methyl.

In certain embodiments, a compound of the present invention is represented by the formula:

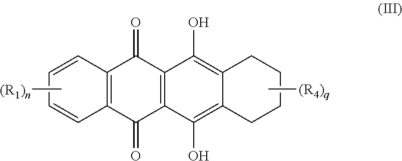

wherein

n is an integer between 0 and 4, inclusive;

q is an integer between 0 and 8, inclusive;

each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —OC(O)R_{A}; —OC(O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₄ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{D}; —C(═O)R_{D}; —CO₂R_{D}; —CN; —SCN; —SR_{D}; —SOR_{D}; —SO₂R_{D}; —NO₂; —N(R_{D})₂; —NHC(O)R_{D}; —OC(O)R_{D}; —OC(O)OR_{D}; or —C(R_{D})₃; wherein each occurrence of R_{D} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, the compound of formula (III) is not of the formula:

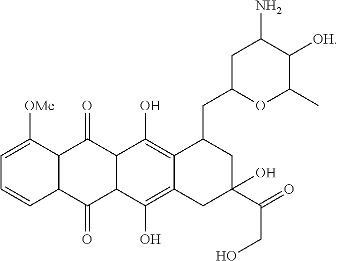

In certain embodiments, the compound of formula (III) is not doxorubicin.

In certain embodiments, the compound of formula (III) is of the formula:

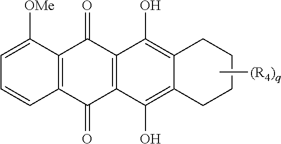

wherein R₄ and q are defined above.

In certain embodiments, the compound of formula (III) is of the formula:

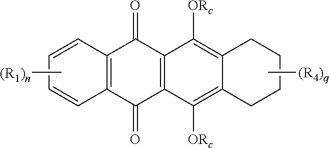

wherein R₁, R_{C}, R₄, n, and q are defined above.

In certain embodiments, the compound of formula (III) is of the formula:

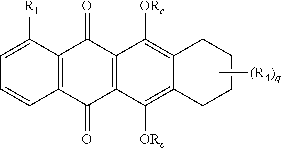

wherein R₁, R_{C}, R₄, and q are defined above.

In certain embodiments, the compound of formula (III) is of the formula:

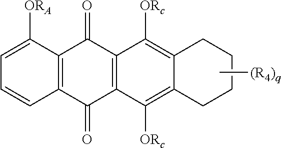

wherein R_{A}, R_{C}, R₄, and q are defined above.

In certain embodiments, the compound of formula (III) is of the formula:

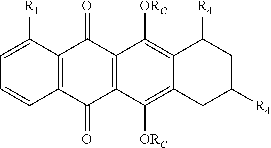

wherein R₁, R_{C}, and R₄, are defined above.

In certain embodiments, a compound of the present invention is represented by the formula:

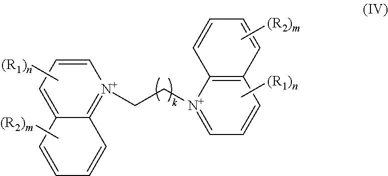

wherein

k is an integer between 0 and 20; inclusive;

each occurrence of n is independently an integer between 0 and 3, inclusive;

each occurrence of m is independently an integer between 0 and 4, inclusive;

each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —OC(O)R_{A}; —OC(O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₂ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{B}; —C(═O)R_{B}; —CO₂R_{B}; —CN; —SCN; —SR_{B}; —SOR_{B}; —SO₂R_{B}; —NO₂; —N(R_{B})₂; —NHC(O)R_{B}; —OC(O)R_{B}; —OC(O)OR_{B}; or —C(R_{B})₃; wherein each occurrence of R_{B} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof. In certain embodiments, m is 0.

In certain embodiments, the compound of formula (IV) is not of the formula:

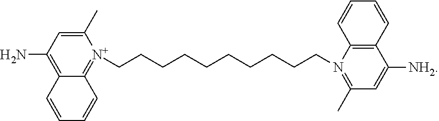

In certain embodiments, the compound of formula (IV) is of the formula:

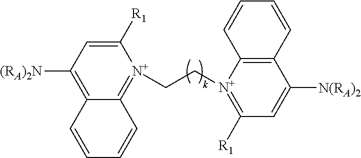

wherein R_{A} and k is defined above.

In certain embodiments, the compound of formula (IV) is of the formula:

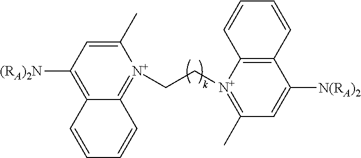

wherein R_{A} and k is defined above.

In certain embodiments, the compound of formula (IV) is of the formula:

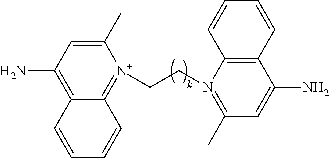

wherein k is defined above.

In certain embodiments, a compound of the present invention is represented by the formula (V):

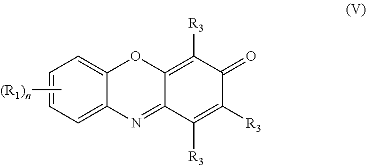

wherein

n is an integer between 0 and 4; inclusive;

each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —OC(O)R_{A}; —OC(O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; —OC(O)R_{C}; —OC(O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

In certain embodiments, the compound of formula (V) is not of the formula:

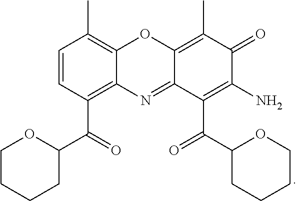

In certain embodiments, the compound of formula (V) is not actinomycin D.

In certain embodiments, the compound of formula (V) is of the formula:

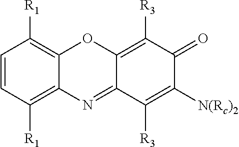

wherein R₁, R₃, and R_{C} are defined above.

In certain embodiments, the compound of formula (V) is of the formula:

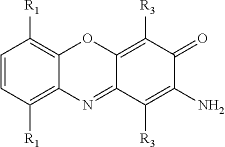

wherein R₁ and R₃ are defined above.

In certain embodiments, the compound of formula (V) is of the formula:

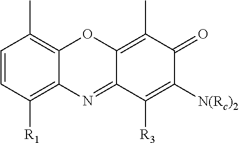

wherein R₁, R₃, and R_{C} are defined above.

In certain embodiments, the compound of formula (V) is of the formula:

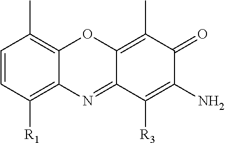

wherein R₁, R₃, and R_{C} are defined above.

It should be understood that, unless otherwise stated, chemical structures or formulae depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of depicted structures or formulae are within the scope of the invention. Unless otherwise stated, all tautomeric forms of the structures or formulae of the invention are within the scope of the invention. Additionally, unless otherwise stated, structures depicted herein are also meant to include compounds that differ only in the presence of one or more isotopically enriched atoms. For example, compounds having the depicted structures or formulae except for the replacement of hydrogen by deuterium or tritium, or the replacement of a carbon by a ¹³C- or ¹⁴C-enriched carbon are within the scope of this invention. Such compounds may be useful, for example, as analytical tools or probes in biological assays.

It should further be understood that the present invention encompasses pharmaceutically acceptable derivatives, and in particular prodrugs, metabolites, and pharmaceutically acceptable salts of the depicted compounds.

In some embodiments, RecA inhibitors of the invention inhibit one or more RecA activities with an IC₅₀ below about 100 μg/ml, 50 μg/ml, 15 μg/ml; 10 μg/ml; 5 μg/ml, 3 μg/ml, or 1 μg/ml. According to the present invention, desirable RecA ATPase inhibitors may even have an IC₅₀ well below 1 μg/ml, or even below 500 ng/ml, 100 ng/ml, 50 ng/ml, 30 ng/ml, 25 ng/ml, 20 ng/ml, 15 ng/ml, 10 ng/ml, 5 ng/ml, 1 ng/ml, or less.

In some embodiments of the present invention, RecA inhibitors are broad spectrum antibiotics in that they inhibit RecA (or the relevant RecA homolog) from more than one different microbial source. In other embodiments, RecA inhibitors have a narrow spectrum activity in that they inhibit one or more activities of RecA (or its relevant homolog) from a specific family of organisms or from a specific organism. In certain embodiments, RecA inhibitors inhibit one or more activities of RecA (or its relevant homolog) from a disease-causing organism (in particular an organism that causes disease in a mammal, e.g., a human). In some embodiments, however, the RecA inhibitors (which may be broad spectrum with regard to microbes) do not inhibit RecA (or the relevant RecA homolog) from one or more higher organisms (e.g., mammals, humans). For example, in some embodiments, RecA inhibitors do not inhibit RAD51.

In some embodiments, the present invention provides RecA inhibitors that inhibit the RecA ATPase activity. For example, the present invention demonstrates that a variety of compounds inhibit RecA ATPase activity in an in vitro luciferase assay (see, for example, Examples 1 and 2). In some embodiments of the present invention, RecA inhibitors that inhibit the RecA ATPase activity do not inhibit certain other cellular ATPases.

In some embodiments, the present invention provides RecA inhibitors that may bind directly to RecA. In some embodiments, RecA inhibitors bind to the RecA ATP binding site. However, in some embodiments, RecA inhibitors do not bind to the RecA ATP binding site (even though they may inhibit the RecA ATPase activity). In certain embodiments, RecA inhibitors bind to two or more different sites on the RecA protein.

Certain RecA inhibitors have been found to potentiate the activity of one or more antibiotic agents (see, International Application No. PCT/US07/03712, filed on Feb. 13, 2007 and International Application No. PCT/US07/03698, filed on Feb. 13, 2007, the contents of all of which are hereby incorporated by reference.). For example, actinomycin D has been found to potentiate the activity of a quinolone antibiotic (ciprofloxacin) as well as that of gentamycin (see, for example, Examples 4 and 5).

Certain RecA inhibitors have been found to reduce the incidence of resistance that may develop to one or more antibiotic agents. RecA inhibitors of the present invention typically have intrinsic antibiotic activity, even in the absence of any other antibiotic agent. See, International Application No. PCT/US07/03712, filed on Feb. 13, 2007 and International Application No. PCT/US07/03698, filed on Feb. 13, 2007, the contents of all of which are hereby incorporated by reference.

In other embodiments, RecA inhibitors can actually protect cells from death. Without wishing to be bound by any particular theory, it is noted that one possible explanation for the present findings is that RecA inhibitors effective for use as antibiotic agents or potentiating agents according to the present invention are those that can simultaneously bind to two distinct sites on RecA, for example to the ATP binding site and to another binding site, including those sites involving R85, F270, Y271, K310, and R32 (see International Application No. PCT/US07/03698, filed on Feb. 13, 2007). Alternatively or additionally, it may be the case that, although many agents can inhibit the RecA ATPase, most such agents also inhibit other ATPases within a cell, causing a variety of stresses and inducing protective mechanisms (e.g., shut down of DNA replication) that allow the cells to avoid the effects of antibiotic agents.

### III—Antibiotic Agents

As discussed herein, RecA inhibitors of the present invention may be used alone or they may be utilized in combination with one or more antibiotic agents.

Exemplary structural classes of antibiotics for use in combination with RecA inhibitors according to the present invention include, but are not limited to, aminoglycosides, aminomethylcyclines, amphenicols, ansamycins, β-lactams (e.g., penicillins or cephalosporins), carbapenems, dapsones, 2,4-diaminopyrimidines, glycopeptides, glycycyclines, ketolides, lincomycins, lincosamides, macrolides, nitrofurans, oxazolidinones, peptides, polymyxins, quinolones, rifabutins, streptogramins, sulfonamides, sulfones, tetracyclines, and combinations thereof.

Exemplary mechanistic classes of antibiotics for use in combination with RecA inhibitors according to the present invention include, but are not limited to, those that inhibit protein synthesis, cell wall synthesis, DNA replication, transcription, and/or cell division. It will be appreciated that biological and biochemical pathways are not mutually exclusive and that some biological or biochemical pathways may be considered to be subsets or sub-pathways of other biological or biochemical pathways. Mechanisms of action more specifically include, but are not limited to, inhibiting protein synthesis (e.g., by binding ribosomal RNA or proteins, blocking tRNA binding to ribosome-mRNA complex, inhibiting peptidyl transferase), inhibiting or interfering with synthesis of a cell wall component (e.g., inhibition of peptidoglycan synthesis, disruption of peptidoglycan cross-linkage, disruption of movement of peptidoglycan precursors, disruption of mycolic acid or arabinoglycan synthesis), cell membrane disruption, inhibiting or interfering with nucleic acid synthesis or processing, acting as “antimetabolites” and either inhibiting an essential bacterial enzyme or competing with a substrate of an essential bacterial enzyme, inhibiting or interfering with cell division.

It is understood by those of ordinary skill in the art that antibiotic agents of a particular structural class typically (though not necessarily) fall within the same mechanistic class.

Examples of antibiotics that can be used in combination with a RecA inhibitor according to the present invention include, but are not limited to bacitracin; cephalosporins (such as cefadroxil, cefazolin, cephalexin, cephalothin, cephapirin, cephradine, cefaclor, cefamandole, cefonicid, ceforanide, cefoxitin, cefuroxime, cefoperazone, cefotaxime, cefotetan, ceftazidime, ceftizoxime, ceftriaxone, and meropenem); cycloserine; fosfomycin, penicillins (such as amdinocillin, ampicillin, amoxicillin, azlocillin, bacamipicillin, benzathine penicillin G, carbenicillin, cloxacillin, cyclacillin, dicloxacillin, methicillin, mezlocillin, nafcillin, oxacillin, penicillin G, penicillin V, piperacillin, and ticarcillin); ristocetin; vancomycin; colistin; novobiocin; polymyxins (such as colistin, colistimathate, and polymyxin B); aminoglycosides (such as amikacin, gentamicin, kanamycin, neomycin, netilmicin, paromomycin, spectinomycin, streptomycin, and tobramycin), tetracyclines (such as demeclocycline, doxycycline, methacycline, minocycline, and oxytetracycline); carbapenems (such as imipenem); monobactams (such as aztreonam); chloramphenicol; clindamycin; cycloheximide; fucidin; lincomycin; puromycin; rifampicin; other streptomycins; macrolides (such as erythromycin and oleandomycin); fluoroquinolones; actinomycin; ethambutol; 5-fluorocytosine; griseofulvin; rifamycins; sulfonamides (such as sulfacytine, sulfadiazine, sulfisoxazole, sulfamethoxazole, sulfamethizole, and sulfapyridine); and trimethoprim. Other antibacterial agents include, but are not limited to, bismuth containing compounds (such as bismuth aluminate, bismuth subcitrate, bismuth subgalate, and bismuth subsalicylate); nitrofurans (such as nitrofurazone, nitrofurantoin, and furozolidone); metronidazole; timidazole; nimorazole; and benzoic acid.

In certain embodiments, RecA inhibitors are used in combination with a quinolone antibiotic. Quinolone antibiotics are compounds that contain a quinolone or a naphthyridine nucleus with any of a variety of different side chains and substituents. Numerous modifications of the originally identified core structures have been made resulting in a large number of compounds with activity against differing groups of bacteria. Quinolone antibiotics are described, e.g., in Ronald and Low (Eds.), “Fluoroquinolone Antibiotics”, Birkhä user Verlag, Basel, 2003; DaSilva et al., Curr. Med. Chem., 2003, 10: 21; Van Bambeke et al., Clin Microbiol. Infect., 2005, 11: 256; U.S. Pat. Nos. 3,669,965; 4,563,459; 4,620,007; 4,382,892; 4,985,557 5,053,407; and 5,142,046).

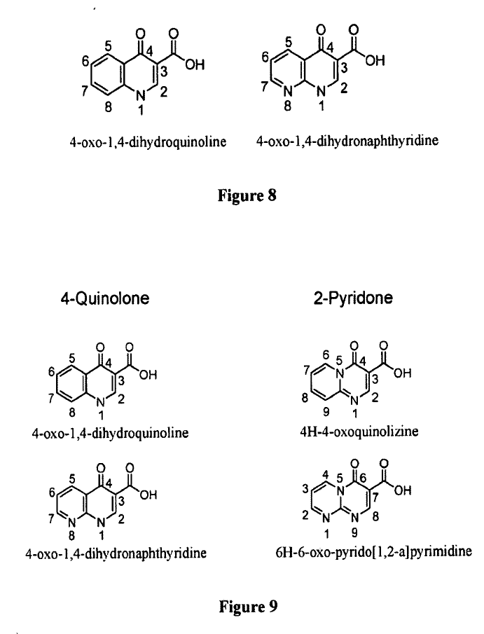

FIG. 8 depicts the core structures and numbering system of classical quinolone antibiotics (4-quinolone and 4-naphthyridine systems). It is noted that the numbering system shown herein is used for purposes of convenience and is not intended to be limiting. The invention encompasses quinolone compounds in which an alternative numbering system is used.

Exemplary quinolone antibiotics include, but are not limited to, any of the antibacterial agents disclosed in the foregoing references including, but not limited to, ciprofloxacin, oxolinic acid, cinoxacin, flumequine, miloxacin, rosoxacin, pipemidic acid, norfloxacin, enoxacin, moxifloxacin, gatifloxacin, ofloxacin, lomefloxacin, temafloxacin, fleroxacin, pefloxacin, amifloxacin, sparfloxacin, levofloxacin, clinafloxacin, nalidixic acid, enoxacin, grepafloxacin, levofloxacin, lomefloxacin norfloxacin, ofloxacin, trovafloxacin, olamufloxacin, cadrofloxacin, alatrofloxacin, gatifloxacin, rufloxacin, irloxacin, prulifloxacin, pazufloxacin, gemifloxacin, sitafloxacin, tosulfloxacin, amifloxacin, nitrosoxacin-A, DX-619, and ABT-492. Quinolone antibiotics include fluoroquinolones (e.g., having a fluorine substituent at the C-6 position), and non-fluoroquinolones. Also included within the scope of quinolone antibiotics are derivatives in which a quinolone is conjugated with, e.g., covalently bound to, another core structure. For example, U.S. Pub. No. 2004-0215017 discloses compounds in which an oxazolidinone, isoxazolinone, or isoxazoline is covalently bonded to a quinolone.

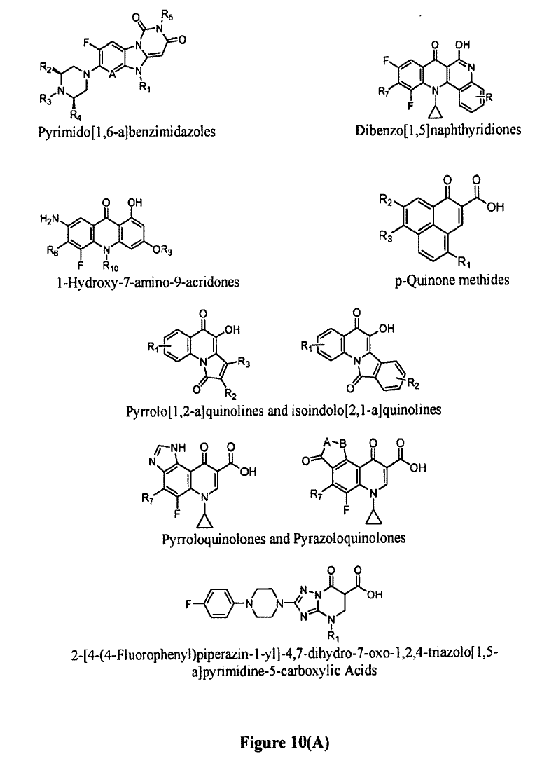

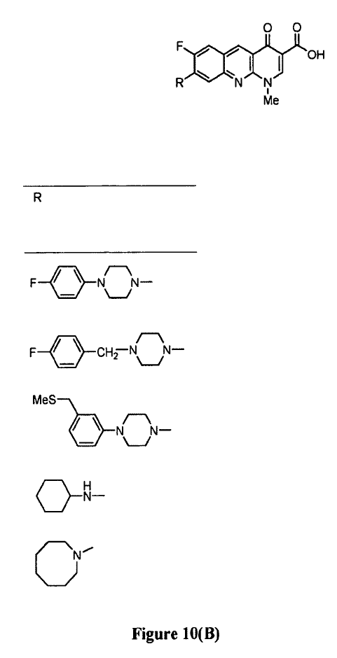

Included within the scope of quinolone antibiotics that can be utilized in accordance with the present invention are compounds that have a core structure related to the 4-oxo-1,4-dihydroquinoline and 4-oxo-1,4 dihydronapthyridine systems, e.g., 2-pyridones, 2-naphthyridinones, and benzo[b]naphthyridones. 2-pyridones are potent inhibitors of bacterial type II topoisomerases (Saiki et al., Antimicrob. Agents Chemother., 1999, 43: 1574). The core structures are depicted in FIG. 9. Also included within the scope of quinolone antibiotics are compounds that have core structures related to the quinolone core structures depicted in FIG. 8 or 9. Certain of these core structures are shown in FIG. 10A or 10B and references thereto are provided in Ronald and Low (Eds.), “Fluoroquinolone Antibiotics”, Birkhä user Verlag, Basel, 2003; DaSilva et al., Curr. Med. Chem., 2003, 10: 21.

### IV—Pharmaceutical Compositions and Kits

The present invention provides pharmaceutical compositions comprising an effective amount of one or more RecA inhibitors of the present invention. In some embodiments, the pharmaceutical compositions further comprise one or more antibiotic agents other than the RecA inhibitors.

Suitable preparations, e.g., substantially pure preparations, of RecA inhibitors and/or antibiotic agents may be combined with pharmaceutically acceptable carriers or excipients, etc., to produce an appropriate pharmaceutical composition. The invention therefore provides a variety of pharmaceutically acceptable compositions for administration to a subject comprising (i) a RecA inhibitor in accordance with the present invention; and (ii) a pharmaceutically acceptable carrier or excipient. The invention further provides a pharmaceutically acceptable composition comprising (i) an inventive RecA inhibitor in accordance with the present invention; (ii) an antibiotic agent; and (iii) a pharmaceutically acceptable carrier or excipient. The invention also provides a pharmaceutically acceptable unit dosage form containing a predetermined amount of a RecA inhibitor, optionally further including a predetermined amount of an antibiotic agent.

Pharmaceutical compositions of the present invention may be provided as immediate release formulations or as sustained release formulations. A variety of strategies are known in the art for achieving sustained release, e.g., by prolonging residence time in the stomach (such as through the use of swellable polymers), providing pH or enzyme-sensitive coatings, employing bioadhesive coatings that stick to the walls of the stomach or intestine, etc. See, e.g., U.S. Pub. No. 2004-0024018 and references therein.

Furthermore, inventive pharmaceutical compositions may be formulated for any desirable route of delivery including, but not limited to, intravenous, intramuscular, by inhalation (e.g., as an aerosol), by catheter, intraocularly, oral, rectal, intradermal, by application to the skin, etc.

It is to be understood that the pharmaceutical compositions of the invention, when administered to a subject, are preferably administered for a time and in an amount sufficient to treat the disease or condition for which they are administered, e.g., a bacterial infection.

In certain embodiments of the pharmaceutical compositions of the invention, one or more of RecA inhibitors of the invention and/or antibiotic agents is provided in the form of a pharmaceutically acceptable derivative (e.g., a prodrug), by which is meant any non-toxic salt, ester, salt of an ester or other derivative of a agent of this invention that, upon administration to a recipient, directly or indirectly provides the relevant RecA inhibitor or antibiotic agent, or an inhibitorily active metabolite or residue thereof. As used herein, the term “inhibitorily active metabolite or residue thereof” means that a metabolite or residue thereof exhibits inhibitory activity towards a protein (e.g., RecA) or microorganism, as appropriate.

As is understood in the art, pharmaceutically acceptable carriers or excipients generally refer to non-toxic components that are appropriate for administration to a subject and that do not destroy the pharmacological activity of the RecA inhibitor(s) and/or antibiotic agent(s) with which they are combined. Appropriate pharmaceutically acceptable carriers or excipients are known in the art and include, for example, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Solvents, dispersion media, coatings, isotonic and absorption delaying agents, and the like, compatible with pharmaceutical administration may be included. Supplementary active agents, e.g., agents independently active against the disease or clinical condition to be treated, or agents that enhance availability and/or activity of an RecA inhibitor or an antibiotic agent, can also be incorporated into the compositions.

Pharmaceutically acceptable salts of RecA inhibitors of the invention and/or antibiotic agents include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining RecA inhibitors or antibiotic agents and their pharmaceutically acceptable acid addition salts.

Salts derived from appropriate bases include alkali metal (e.g., sodium and potassium), alkaline earth metal (e.g., magnesium), ammonium and N(C1-4 alkyl)4 salts. This invention also envisions the quaternization of any basic nitrogen-containing groups of the compounds disclosed herein. Water or oil-soluble or dispersible products may be obtained by such quaternization.

A pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), phosphate buffered saline (PBS), or Ringer's solution.

Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

It is generally desirable that pharmaceutical compositions, particularly for injection, be sterile, if possible, and should be fluid to the extent that easy syringability exists.

Pharmaceutical formulations are desirably stable under the conditions of manufacture and storage and should be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. A desired fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of injectable compositions can be brought about by including in the composition an agent which delays absorption, for example, aluminum monostearate and gelatin. Prolonged absorption of oral compositions can be achieved by various means including encapsulation.

Sterile injectable solutions can be prepared by incorporating the active agent(s) (i.e., RecA inhibitor(s) of the invention and/or antibiotic agent(s)) in a required amount in an appropriate solvent with one or a combination of ingredients enumerated above, as required, followed by filtered sterilization. Preferably solutions for injection are free of endotoxin. Generally, dispersions are prepared by incorporating the active agent(s) into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active agent(s) can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, inventive compositions may desirably be delivered in the form of an aerosol spray from a pressured container or dispenser which contains a suitable propellant, e.g., a gas such as carbon dioxide, or a nebulizer. Liquid or dry aerosol (e.g., dry powders, large porous particles, etc.) can be used. The present invention also contemplates delivery of compositions using a nasal spray. Pharmaceutical compositions to be administered by nasal aerosol or inhalation may be prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

For topical applications, inventive pharmaceutical compositions may be formulated in a suitable ointment containing the active component suspended or dissolved in one or more carriers. Carriers for topical administration of the agents of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For local delivery to the eye, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Inventive pharmaceutical compositions may be formulated for transmucosal or transdermal delivery. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams as generally known in the art.

Inventive pharmaceutical compositions may be formulated as suppositories (e.g., with conventional suppository bases such as cocoa butter and other glycerides) or as retention enemas for rectal delivery.

In some embodiments, inventive pharmaceutical compositions include one or more agents intended to protect the active agent(s) against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polyethers, and polylactic acid. Methods for preparation of such formulations will be apparent to those skilled in the art. Certain of the materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811 and other references listed herein. Liposomes, including targeted liposomes (e.g., antibody targeted liposomes) and pegylated liposomes have been described (C. B. Hansen et al., Biochim. Biophys. Acta, 1995, 1239: 133-144; V. P. Torchilin et al., Biochim. Biophys. Acta, 2001, 1511: 397-411; T. Ishida et al., FEBS Lett., 1999, 460: 129-133).

It is often desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with the required pharmaceutical carrier.

In general, pharmaceutical compositions are formulated to contain an amount of active agent(s) (i.e., RecA inhibitors and/or antibiotic agents) sufficient to achieve a desired biological or pharmacological effect while minimizing any associated toxicity. Toxicity and therapeutic efficacy of active agents can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents which exhibit high therapeutic indices are preferred. While agents that exhibit toxic side effects can be used, care should be taken to design a delivery system that targets such agents to the site of affected tissue in order to minimize potential damage to uninfected cells and, thereby, reduce side effects.

The data obtained from cell culture assays and animal studies can be used in formulating a range of dosage for use in humans. The dosage of such agents lies preferably within a range of circulating concentrations that include the ED₅₀ with little or no toxicity. The dosage can vary within this range depending upon the dosage form employed and the route of administration utilized. For any agent used in accordance with the present invention, the therapeutically effective amount (e.g., the amount that is therapeutically effective to achieve a desired degree of antibiotic activity) can typically be estimated initially from cell culture assays. However, it is generally more desirable to establish dosing based on studies in animal models, where amounts required to achieve a circulating plasma concentration range that includes the IC₅₀ (e.g., the concentration of the test agent which achieves a half-maximal inhibition of symptoms, half-maximal inhibition of growth or survival of an infectious agent, etc.) can be determined. Such information can be used to more accurately determine useful doses in humans. Levels in plasma can be measured, for example, by high performance liquid chromatography.

A therapeutically effective amount of an active agent in a pharmaceutical composition typically ranges from about 0.001 to about 100 mg/kg body weight, about 0.01 to about 25 mg/kg body weight, about 0.1 to about 20 mg/kg body weight, about 1 to about 10 mg/kg, about 2 to about 9 mg/kg, about 3 to about 8 mg/kg, about 4 to about 7 mg/kg, or about 5 to about 6 mg/kg body weight. Other exemplary doses include, for example, about 1 μg/kg to about 500 mg/kg, about 100 μg/kg to about 5 mg/, about 1 μg/kg to about 50 μg/kg). In general, smaller doses are typically required for local administration as contrasted with systemic administration. Furthermore, it is understood by those of ordinary skill in the art that appropriate doses in any particular circumstance depend upon the potency of the agent(s) utilized, and may optionally be tailored to the particular recipient, for example, through administration of increasing doses until a preselected desired response is achieved. It is particularly understood that the specific dose level for any particular subject may depend upon a variety of factors including the activity of the specific agent(s) employed, the age, body weight, general health, gender, and diet of the subject, the time of administration, the route of administration, the rate of excretion, etc.

Inventive pharmaceutical compositions may be administered according to any desired schedule, typically selected to achieve optimal therapeutic effect. For instance, inventive pharmaceutical compositions can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, etc. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with an inventive composition can include a single treatment or, in many cases, can include a series of treatments. It will be appreciated that a range of different dosage combinations (e.g., doses of RecA inhibitor and/or antibiotic agent) can be used.

The present invention also provides pharmaceutical packs or kits comprising one or more containers (e.g., vials, ampoules, test tubes, flasks, or bottles) containing one or more ingredients of the inventive pharmaceutical compositions, for example, allowing for the simultaneous or sequential administration of the RecA inhibitors and antibiotic agent(s). Optionally associated with such container(s) can be a notice in the form prescribed by a governmental agency regulating the manufacture, use or sale of pharmaceutical products, which notice reflects approval by the agency of manufacture, use or sale for human administration. Different ingredients may be supplied in solid (e.g., lyophilized) or liquid form. Each ingredient will generally be suitable as aliquoted in its respective container or provided in a concentrated form. Kits may also include media for the reconstitution of lyophilized ingredients. The individual containers of the kit are preferably maintained in close confinement for commercial sale.

### IV—Use of Rec A Inhibitors

In general, according to the present invention, inhibition of RecA has several important effects on cells. First, cells in which RecA is inhibited have a reduced ability to repair damage to their DNA, and also have a reduced ability to infect a host. Thus, inhibition of RecA reduces pathogen virulence.

In particular, cells (e.g., those of an infectious organism) in which RecA is inhibited are particularly susceptible to the oxidative burst that occurs within phagocytes. Thus, when microbial organisms and/or pathogens are ingested by phagocytes, they are exposed to a variety of lethal factors, including oxidative radicals, acidic pH, and destructive enzymes. Oxidative radicals in particular cause DNA damage. Cells with reduced RecA activity have reduced ability to recover from such DNA damage and therefore succumb more readily to phagocytotic attack.

Furthermore, many types of microbial infections are initiated by the adherence of pathogens to host tissues. This process requires interaction of microbial surface proteins called adhesions with proteins, such as fibronectin, on the surface of host cells. RecA has been shown to participate in the fibronectin-binding pathway in some bacteria (e.g., S. aureus; Bisognano et al., J. Biol. Chem., 2004, 279: 9064). Thus, inhibition of RecA can also reduce virulence by reducing adhesion of microbial cells to host cells.

Thus, the present invention provides RecA inhibitors that are useful in the treatment of microbial infection. Such RecA inhibitors may be utilized alone (i.e., in the absence of any other antibiotic agent) to treat infection, or alternatively may be utilized together with one or more other antibiotic agents. In certain embodiments, the combination of an antibiotic agent with a RecA inhibitor for the treatment of microbial infection allows the antibiotic agent to be used at a dose below its convention dose, and/or on a less frequent dosing schedule. This can be advantageously used to reduce drug toxicity observed with certain antibiotic agents.

Inventive RecA inhibitors are useful to inhibit growth of a wide variety of microbial types including, for example, gram negative bacteria, gram positive bacteria and/or acid fast bacteria. Particular examples of bacteria whose growth or proliferation can be inhibited include, but are not limited to, members of the following genuses: Actinomyces, Staphylococcus, Streptococcus, Enterococcus, Erysipelothrix, Neisseria, Branhamella, Listeria, Bacillus, Corynbacterium, Erysipelothrix, Gardnerella, Mycobacterium, Nocardia, Enterobacteriaceae, Escherichia, Salmonella, Shigella, Yersinia, Enterobacter, Klebsiella, Citrobacter, Serratia, Providencia, Proteus, Morganella, Edwardsiella, Erwinia, Vibrio, Aeromonas, Helicobacter, Campylobacter, Eikenella, Pasteurella, Pseudomonas, Burkholderia, Stenotrophomonas, Acinetobacter, Ralstonia, Alcaligenes, Moraxella, Mycoplasma, Legionella, Francisella, Brucella, Haemophilus, Bordetella, Clostridium, Bacteroides, Porphyromonas, Prevotella, Fusobacterium, Borrelia, Chlamydia, Rickettsia, Ehrlichia, Bartonella, Trichomonas, Treponema, and combinations thereof (i.e., infections established by more than one bacterial strain).

In particular embodiments of the invention the bacteria are species that are causative agents of disease in humans and/or animals. Examples include, but are not limited to, Aeromonas hydrophila, Bacillus subtilis, Escherichia coli, Enterobacter cloacae, Campylobacter jejuni, Haemophilus influenzae, Klebsiella pneumoniae, Klebsiella oxytoca, Legionella pneumophila, Pasteurella multocida, Proteus mirabilis, Proteus vulgaris, Morganella morganii, Helicobacter pylori, Neisseria gonorrhoeae, Pseudomonas aeruginosa, Salmonella enterica, Salmonella typhimurium, Staphylococcus aureus, Staphylococcus epidermidis, Streptococcus pneumoniae, Streptococcus pyogenes, Streptococcus agalactiae, and combinations thereof.

In certain embodiments of the invention, the bacterial species or strain is one that is sensitive to a particular antibiotic agent or class of antibiotic agents.

In certain embodiments of the invention, the bacterial species or strain is one that is resistant to a particular antibiotic agent or class of antibiotic agents.

RecA inhibitors of the invention and compositions containing them can be used to inhibit microbial growth and/or survival in a variety of contexts. For example, they may be employed to inhibit growth and/or survival of organisms maintained in cell culture or inhabiting locations in the environment, e.g., inert surfaces, clothing, towels, bedding, utensils, etc. Of particular interest are fomites, i.e., inanimate objects that may be contaminated with disease-causing microorganisms and may serve to transmit disease to a human or animal. Such locations or objects can be contacted with a solution containing a RecA inhibitor, and optionally including one or more other antibiotic agents. RecA inhibitors of the invention, alone or together with one or more other antibiotic agents, can be added to food or water, particularly for the prevention of microbial disease in animals.

A RecA inhibitor may be administered alone or in combination with another antibiotic agent, may be administered to a subject in need thereof, e.g., a human or animal suffering from or at risk of a bacterial infection. When administered in combination, the RecA inhibitor of the invention and antibiotic, may be components of a single pharmaceutical composition or may be administered as individual pharmaceutical compositions. They may be administered using the same route of administration or different routes of administration. In certain embodiments of the invention a unit dosage form containing a predetermined amount of a RecA inhibitor and optionally a predetermined amount of another antibiotic agent is administered. It will be appreciated by those of ordinary skill in the art that the predetermined about of RecA inhibitor may be different, even for the same RecA inhibitor, if it is being administered alone or in combination. In certain embodiments, the RecA inhibitor and the one or more additional antibiotic agent(s) are administered concomitantly. In other embodiments, the RecA inhibitor and the antibiotic agent(s) are administered sequentially. For example, in certain embodiments, the RecA inhibitor is administered prior to administration of the antibiotic agent(s).

A therapeutic regimen that includes a RecA inhibitor and another antibiotic agent may (i) allow the use of a reduced daily dose of the antibiotic without significantly reducing efficacy; (ii) allow the use of a shorter course of administration of the antibiotic without significantly reducing efficacy; or both.

Infections and infection-related conditions that can be treated using a RecA inhibitor of the invention (and optionally another antibiotic agent) include, but are not limited to, pneumonia, meningitis, sepsis, septic shock, sinusitis, otitis media, mastoiditis, conjunctivitis, keratitis, external otitis (e.g., necrotizing otitis externa and perichondritis), laryngeal infections (e.g., acute epiglottitis, croup and tuberculous laryngitis), endocarditis, infections of prosthetic valves, abscesses, peritonitis, infectious diarrheal diseases, bacterial food poisoning, sexually transmitted diseases and related conditions, urinary tract infections, pyelonephritis, infectious arthritis, osteomyelitis, infections of prosthetic joints, skin and soft tissue infections, oral infections, dental infections, nocardiosis and actinomycosis, mastitis, brucellosis, Q fever, anthrax, wound infections, etc.

In certain embodiments of the invention, a RecA inhibitor, optionally together with another antibiotic agent, is used to treat or prevent infection associated with an indwelling device. Indwelling devices include surgical implants, prosthetic devices, and catheters, i.e., devices that are introduced to the body of an individual and remain in position for an extended time. Such devices include, for example, artificial joints, heart valves, pacemakers, defibrillators, vascular grafts, vascular catheters, cerebrospinal fluid shunts, urinary catheters, continuous ambulatory peritoneal dialysis (CAPD) catheters, spinal rods, implantable pumps for medication delivery, etc. RecA inhibitors (and other antibiotic agents) can be applied to, coated on, imbedded in, or otherwise combined with an indwelling device to prophylactically treat (e.g., delay the onset of, reduce the severity of, or prevent entirely) infections. Alternatively or additionally, a RecA inhibitor (optionally together with another antibiotic agent) of the invention may be administered to a subject to achieve a systemic effect shortly before, or concomitantly with, insertion of an indwelling device. Of course, local delivery of a RecA inhibitor and/or antibiotic may also be employed. Furthermore, any RecA inhibitor and antibiotic agent may be delivered separately (e.g., by different routes and/or at different times). Treatment with inventive RecA inhibitor and/or another antibiotic agent may be continued after implantation of the device, during all or part of the time during which the device remains in the body, and, optionally, thereafter. Alternatively or additionally, RecA inhibitors (and other antibiotic agents) can be used to bathe an indwelling device immediately before insertion and/or to bathe wounds or sites of insertion. Exemplary concentrations useful for these purposes typically range between about 1 μg/ml and 10 μg/ml.

RecA inhibitors (and other antibiotic agents) of this invention may be used before, during, or after dental treatment or surgery.

Any of a variety of methods may be employed to identify a subject in need of treatment (e.g., a subject at risk of or suffering from a microbial infection) according to the present invention. For example, such methods include clinical diagnosis based at least in part on symptoms, imaging studies, immunodiagnostic assays, nucleic acid based diagnostics, and/or isolation and culture of potentially causative microorganisms from samples, such as blood, urine, sputum, synovial fluid, cerebrospinal fluid, pus, or any sample of body fluid or tissue.

Inventive methods can include a step of identifying a subject suffering from or at risk of a microbial infection, a step of identifying a microorganism suspected of causing the infection, a step of selecting a therapeutic regimen based at least in part on the identity or suspected identity of the microorganism and/or the location or characteristics of the infection. In certain embodiments of the invention, the method includes determining that a subject has a significant likelihood (e.g., at least 5%) of suffering from or being at risk of infection by a microorganism that is resistant to one or more antibiotics.

A subject is “at risk of” an infection in any of a variety of circumstances. “At risk of” implies at increased risk of, relative to the risk such subject would have in the absence of one or more circumstances, conditions, or attributes of that subject, and/or (in the case of humans) relative to the risk that an average, healthy member of the population would have. Specific examples of conditions that place a subject “at risk” include, but are not limited to, immunodeficiencies (particularly those affecting the humoral or non-specific (innate) immune system); prior treatment with antibiotic agent(s) that may have reduced or eliminated normal microbial flora; treatment with agent(s) that suppress the immune system (e.g., cancer chemotherapy, immunosuppressive agents); chronic diseases such as diabetes or cystic fibrosis; surgery or other trauma; infancy or old age; occupations, events, or living conditions that entail exposure to pathogenic microorganisms; etc.

While it is anticipated that RecA inhibitors will find particular use for inhibiting the growth and/or survival of microorganisms, they may also be used for other purposes. For example, although many embodiments of the invention utilize RecA inhibitors that do not inhibit activities of higher organism homologs (e.g., RAD51), in some embodiments of the invention, it may be desirable to utilize RecA inhibitors that do act against homologs in higher organisms. For example, such RecA inhibitors are useful specifically in order to treat disorders, diseases or defects associated with activity of such higher organism homologs, and/or to potentiate or reduce resistance to other agents being used to treat a relevant disorder, disease, or defect. Indeed, in some embodiments of the invention, it may be desirable to utilize RAD51 inhibitors that do not also inhibit RecA.

To give but one particular example, RecA inhibitors that inhibit RAD51 and/or other mammalian topoisomerases may be useful in the treatment of any of a variety of cancers, either alone or in combination with one or more other anti-cancer agents. In some embodiments, RecA inhibitors are utilized in an amount effective to have anti-cancer activity, to potentiate anti-cancer activity of another agent, to reduce resistance to another agent, or combinations thereof. For instance, other agents that could be used in combination with RecA inhibitors in the treatment of cancer include, but are not limited to alkylating agents, topoisomerase I inhibitors, topoisomerase II inhibitors, RNA/DNA antimetabolites, DNA antimetabolites, antimitotic agents, natural antineoplastic agents, hormonal antineoplastic agents, angiogenesis inhibitors, differentiating agents, gene therapy agents, biological response modifiers, anti-metastatic agents, and combinations thereof.

In some embodiments of the invention, RecA inhibitors are used to treat cancer in subjects receiving radiation therapy.

### EXAMPLES

### Example 1

### High Throughput RecA-Luciferase Assay

An in vitro RecA-luciferase assay was used to screen for RecA inhibitors. RecA is a DNA-dependent ATPase (i.e., it catalyzes the reaction adenosine triphosphate [ATP]→adenosine diphosphate [ADP]). An assay was developed for RecA ATPase activity based on detection of the amount of ATP remaining in a reaction mixture following incubation of RecA protein, DNA, and ATP. In this example, E. coli RecA protein (Epicentre, Madison, Wis.) was incubated in reaction buffer with DNA, ATP, and any test compound or compounds for a measured amount of time. The quantity of ATP remaining after the RecA reaction was measured by a subsequent luciferase assay. The luciferase emit more light as more ATP was available.

For high throughput screening, the following reagents were added to each well of a 384 well plate:

| Reagents | Volume | |
|---|---|---|
| 10 X PNK buffer | 2.5 | μL |
| RecA Epicentre (5 mg/ml) | 0.1 | μL |
| Compound of interest | 0.1 | μL |
| H2O | 22.175 | μL |
| M13 DNA | 0.125 | μL |
| Total | 25 | μL |

The reagents were mixed and incubate at room temperature for about 5 minutes. Then, 5.0 μL of ATP mix, as indicated below, were added to each well.

| Reagents | Volume | |
|---|---|---|
| 10 X PNK buffer | 0.5 | μL |
| ATP 1 mM | 0.018 | μL |
| H2O | 4.482 | μL |
| Total | 5.0 | μL |

The mix was incubated at room temperature for about 90-120 minutes. 12.5 μL of Kinase-Glo® plus (Promega) were added to each well and incubated at room temperature for about 10 minutes or more. The luminescence of the reaction was measured.

A positive control (having no DNA added to the well) and negative control (having DNA and no compounds) were run with each screen. Positive responses were based upon the percent reduction of activity of luciferase, with a high percentage representing increased RecA inhibition. Percent inhibition can be calculated according to the following equation:

$$\frac{( RLU in presence of compound ) - ( RLU in DMSO )}{( RLU in absence of DNA ) - ( RLU in DMSO )} .$$

### Example 2

### Identification of RecA Inhibitors Using RecA Luciferase Assay

More than 22,000 compounds were tested using the high throughput RecA-luciferase assay described in Example 1. Some of the tested compounds were from the NERCE collection of compounds (http://iccb.med.harvard.cdu) at least including the following 4 libraries.

### ASINEX

ASINEX library contains 36 plates; but we screened the first 35 including plates 1671 to 1705. Compounds are at a concentration of 5 mg/ml in DMSO. This collection was purchased by the New England Regional Center of Excellence in Biodefense and Emerging Infectious Diseases (NERCE/BEID) in February 2006. These compounds were specifically chosen to increase the overall diversity of the ICCB-Longwood collection, as well as for favorable physico-chemical properties such as solubility, decreased toxicity, and increased stability.

### CHEMDIV 4

CHEMDIV 4 library contains 14,677 compounds in 40 plates. We screened plates 1607 to 1635. Compounds are at a concentration of 5 mg/ml in DMSO. This collection was purchased by the New England Regional Center of Excellence in Biodefense and Emerging Infectious Diseases (NERCE/BEID) in February 2006. These compounds were specifically chosen to increase the overall diversity of the ICCB-Longwood collection, as well as for favorable physico-chemical properties such as solubility, decreased toxicity, and increased stability.

### BIOMOL ICCB-Longwood Known Bioactives 2

This library contains about 480 compounds in two plates: 1791 and 1792. Concentration for each compound is about 5 mg/ml (˜13 mM) for most compounds; or 0.5 mg/ml (˜1.3 mM) for “potent” compounds. This collection was purchased in June 2006 from BIOMOL (catalogue #2840; WWW.BIOMOL.COM) and plated in October 2006. It is based on the ICCB-Longwood Bioactives 1 collection (PL-0684-0685). This collection is also identical to the compounds that were in the high concentration of plates 1361-1362. It was assembled by BIOMOL, working together with the ICCB-Longwood, to provide a library of compounds that affect a wide variety of biological pathways relevant to ICCB-Longwood screening projects. The collection includes many classes of compounds containing ion channel blockers, GPCR ligands, second messenger modulators, nuclear receptor ligands, actin and tubulin ligands, kinase inhibitors, protease inhibitors, gene regulation agents, lipid biosynthesis inhibitors, as well as other well-characterized compounds that perturb cell pathways. We hope that this collection will help both in assay development and in target identification.

This collection contains 41 FDA-approved drugs. However, investigators wishing to screen the maximum number of FDA-approved drugs can achieve approximately 46% coverage (561 compounds) of the FDA set by screening both NINDS and Prestwick. Including the ICCB Biomol Known Bioactives will increase coverage to ˜47% of the FDA set, as there is some redundancy among these three libraries.

### NIBR1

This library has 44,000 compounds. We screened only five plates [1796 to 1799] equivalent to about 1760 compounds. Compounds are at 10 mM in DMSO. The NIBR1 library was provided by the Novartis Institutes for Biomedical Research Academic HTS Partnership Program. It is a select set of 44,000 public domain compounds. This fully annotated, high quality collection was selected by drug discovery experts to provide drug-like qualities, chemical diversity, and potential for structure activity relationship follow-up experiments.

A summary of exemplary positive screening results are provided in Table 1.

TABLE 1
Exemplary positive screening results
Experiment 1

| POSITIVE | NO DNA | 36709009 | | | |
|---|---|---|---|---|---|
| NEGATIVE | plus DNA | 14797912 | | 40% activity | |
| | PLATE | WELL | LUMEN | % ACTIVITY | |
| | 1791 | F8 | 29532660 | 80 | |
| | 1791 | F8 | 33392090 | 90 | 300 nl of compound |
| | 1791 | O14 | 25992870 | 70 | 300 nl of compound |
| | 1791 | H15 | 27710850 | 75 | 300 nl of compound |
| | 1791 | O22 | 24527230 | 66 | 300 nl of compound |

Experiment 2

| POSITIVE | NO DNA | 31660225 | | |
|---|---|---|---|---|
| NEGATIVE | plus DNA | 11217911 | | 35.4% activity |
| | PLATE | WELL | LUMEN | % ACTIVITY |
| | 1568 | P18 | 25643980 | 80.99 |
| | 1569 | G4 | 33371700 | 105.4 |
| | 1569 | L10 | 3.57E+08 | 112.81 |
| | 1569 | M11 | 30612690 | 96.69 |
| | 1569 | B15 | 31049920 | 98.07 |
| | 1569 | M17 | 25641260 | 80.98 |
| | 1569 | G18 | 32562960 | 102.85 |
| | 1570 | O6 | 30943990 | 97.73 |
| | 1920 | I3 | 25672980 | 81.08 |
| | 1920 | D7 | 28993650 | 91.57 |
| | 1920 | N16 | 27005920 | 83.06 |
| | 1921 | K15 | 26297820 | 83.06 |
| | 1921 | I16 | 23416410 | 73.96 |
| | 1921 | K20 | 25362940 | 80.1 |
| | 1922 | I8 | 24068220 | 76.02 |
| | 1922 | G11 | 31916270 | 100.8 |
| | 1923 | I9 | 27640540 | 87.3 |
| | 1792 | I3 | 26938210 | 85.08 |
| | 1792 | C13 | 26153200 | 82.6 |
| | 1792 | D19 | 26142760 | 82.57 |
| | 1792 | P21 | 24806240 | 78.35 |

Experiment 3
NIBR

| POSITIVE | no DNA | 33139939 | | |
|---|---|---|---|---|
| NEGATIVE | plus DNA | 14662693 | | %44.24 activity |
| | PLATE | WELL | LUMEN | % ACTIVITY OF LUCIFERASE |
| | 1795 | K1 | 22214900 | 67.03 |

Experiment 4
CHEMDIV and ASINEX

| POSITIVE | NO DNA | 35249208 | | 100% |
|---|---|---|---|---|
| NEGATIVE | plus DNA | 11430796 | | 32.4% ACTIVITY |
| | PLATE | WELL | LUMEN | % ACTIVITY OF LUCIFERASE |
| | 1609 | O17 | 21280940 | 60.37 |
| | 1610 | B22 | 28319680 | 80.34 |
| | 1610 | E7 | 31339700 | 88.9 |
| | 1613 | A16 | 28005880 | 79.45 |
| | 1616 | D1 | 22853660 | 64.83 |
| | 1618 | H10 | 20088040 | 56.98 |
| | 1619 | J15 | 22278360 | 63.2 |
| | 1624 | H18 | 17994760 | 50.05 |
| | 1625 | N22 | 17718320 | 50.26 |
| | 1629 | G3 | 23376900 | 66.31 |
| | 1630 | G7 | 18498960 | 52.48 |
| | 1634 | D2 | 19842640 | 56.29 |
| | 1634 | D4 | 19558280 | 55.48 |
| | 1634 | D6 | 19344300 | 54.87 |
| | 1634 | D8 | 19271720 | 54.67 |
| | 1634 | D10 | 19425440 | 55.1 |
| | 1634 | F2 | 20270100 | 57.5 |
| | 1634 | F4 | 19712160 | 55.92 |
| | 1634 | F6 | 19685820 | 55.84 |
| | 1634 | F8 | 19501060 | 55.32 |
| | 1634 | F10 | 19945680 | 56.58 |
| | 1634 | F12 | 19367600 | 54.94 |
| | 1673 | G20 | 31125600 | 88.3 |
| | 1678 | I10 | 27210020 | 77.19 |
| | 1682 | O5 | 21995820 | 62.4 |
| | 1683 | A3 | 21713200 | 61.59 |
| | 1687 | B10 | 21167800 | 60.05 |
| | 1693 | B16 | 21183360 | 60.09 |
| | 1696 | A15 | 18581120 | 52.71 |
| | 1696 | E9 | 18335000 | 52.01 |
| | 1697 | C2 | 27167760 | 77.07 |
| | 1700 | L6 | 28076900 | 79.65 |
| | 1705 | L18 | 20298180 | 57.58 |

FIGS. 1 and 2 present representative structures of 59 particularly active compounds identified from the screen. These exemplary compounds include, but are not limited to, quinacrine dihydrochloride dihydrate, daunorubicin hydrochloride, ellipticine, mitoxantrone dihydrochloride, coralyne chloride hydrate, dequalinium dichloride, doxorubicin hydrochloride, propidium iodide, hexachlorophene, bithionol, quinacrine hydrochloride, acrisorcin (Aminacrine), acriflavinium hydrochloride, coralyne chloride, homidium bromide, dequalinium chloride, mitoxathrone hydrochloride, actinomycin D (Dactinomycin).

One compound in particular (Plate 1791, Well F08, ICCB-00687896), demonstrated 80% activity in the RecA-luciferase assay and was selected for further study. This compound is commonly known as actinomycin D.

### Example 3

### Confirming RecA ATPase Inhibition Activity of Actinomycin D

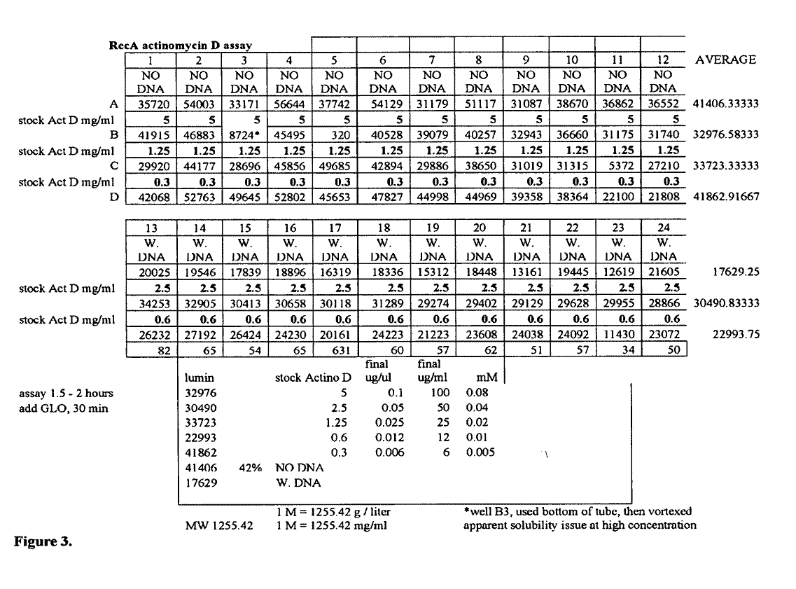

Actinomycin D was further evaluated for its RecA inhibitory activity in an in vitro RecA ATPase assay. Several different concentrations of actinomycin D were tested (see FIG. 3). As illustrated in FIG. 3, actinomycin D effectively inhibited RecA ATPase activity at a low concentration of 0.01 mM (i.e., 12 μg/mL).

### Example 4

### Actinomycin D Potentiates Antibiotic Activity in Cell Growth Assays

The ability of actinomycin D to potentiate antibiotic activity was demonstrated in cell growth assays.

Cell Growth Assay. This assay measures the ability of cells to grow in the presence of a sublethal dose (i.e., a dose below the MIC) of an antibiotic agent (or test agent, or combination thereof). In general, cells are grown, typically to saturation, and are then diluted and inoculated onto rich medium and onto medium containing a sublethal dose of antibiotic agent. Plates are then grown overnight, and OD₆₀₀ is measured after overnight growth, as compared with blank plate. If desired, a test agent can be added to the plate, either alone or in combination with the known antibiotic agent, and the ratio of OD₆₀₀ in the presence of the test agent to OD₆₀₀ in the absence of the test agent, can be determined. Additional ratios that can be useful as controls include, for example, OD₆₀₀ in the presence vs. absence of antibiotic agent; OD₆₀₀ in the absence of any agent vs. in the presence of both antibiotic agent and test agent; etc.

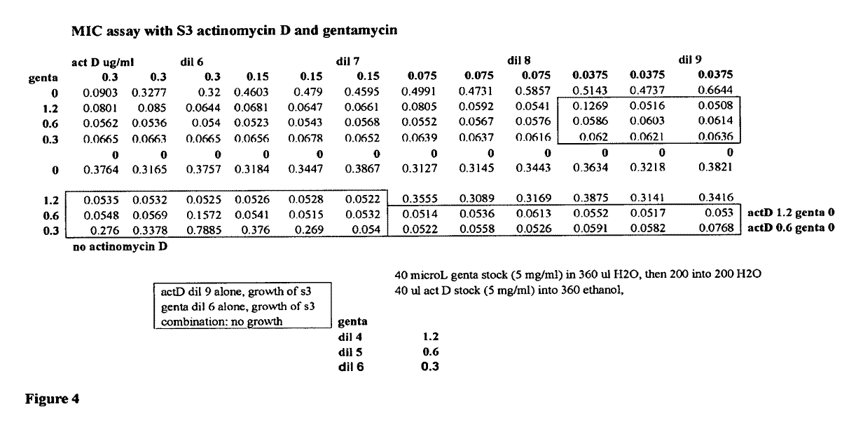

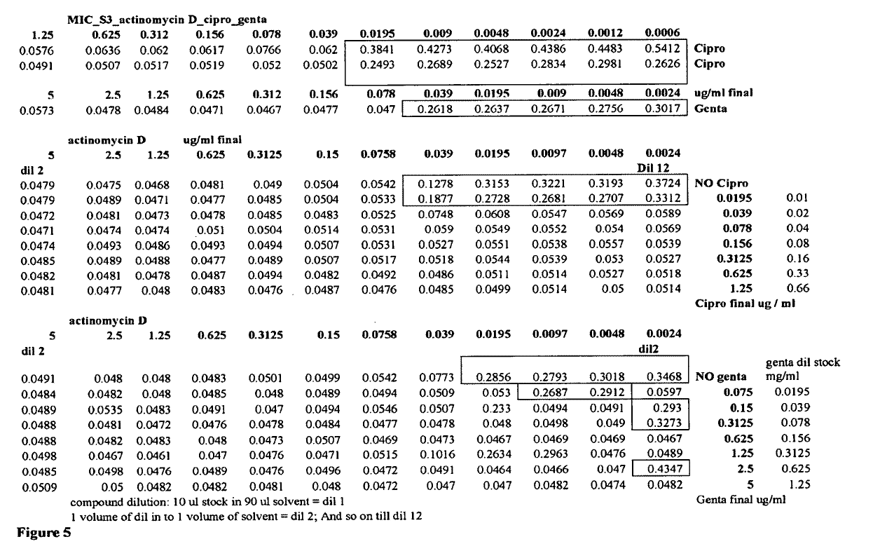

Actinomycin D Potentiates Gentamycin: In the first assay, S. aureus strain S3 was used in a cell growth assay as described in FIG. 4. The strain was grown in the presence and absence of gentamycin and actinomycin D. Various concentrations of gentamycin and actinomycin D were used. Specifically, 40 μL gentamycin stock (5 mg/mL) was first diluted in 360 μL H2O. Then, 200 μL of the diluted gentamycin solution was further mixed with 200 μL H2O. This solution was further diluted to various concentrations, e.g., 1.2 μg/mL (dil 4), 0.6 μg/mL (dil 5), 0.3 μg/mL (dil 5). 40 μL actinomycin D stock (5 mg/mL) was mixed with 360 μL ethanol and was further diluted to various concentrations, e.g., 0.3 μg/mL (dil 6), 0.15 μg/mL (dil 7), 0.075 μg/mL (dil 8), 0.0375 μg/mL (dil 9). Growth inhibition was demonstrated using sub-lethal concentrations of gentamycin and low doses (less than 0.04 ug/ml) of actinomycin D (see the shaded columns in FIG. 4). For example, S. aureus strain S3 grew well in the presence of a low concentration of actinomycin D (0.0375 ug/ml). S. aureus strain S3 grew equally well in the presence of a sublethal concentration of gentamycin (0.3 μg/mL). However, the growth of S. aureus strain S3 was significantly inhibited in the presence of a combination of a sub-lethal concentration of gentamycin (0.3 μg/mL) and a low dose of actinomycin D (0.0375 ug/ml) (see the shaded columns in FIG. 4). Therefore, actinomycin D clearly potentiates gentamycin. The ability of actinomycin D to potentiate gentamycin was further confirmed in the second assay as shown in FIG. 5.

Actinomycin D potentiates ciprofloxacin: In the second assay, S. aureus was also grown in the presence and absence of ciprofloxacin and actinomycin D as described in FIG. 5. Growth inhibition was demonstrated using sub-lethal concentrations of ciprofloxacin and low doses (less than 0.04 ug/ml) of actinomycin D (see the shaded columns in FIG. 5).

### Example 5

### Actinomycin D Analogues

Having identified actinomycin D as a particularly potent RecA inhibitor and antibiotic potentiator for use in accordance with the present invention, the present Applicant contemplated that related compounds are likely to share some or all of actinomycin D's activities. Such related compounds may include various actinomycin D analogues or derivatives known in the art. FIG. 6 illustrates exemplary analogues of actinomycin D that can be used in accordance with the present invention. Additional actinomycin D analogues are described in U.S. Pat. Nos. 4,514,330, 4,680,382, 4,966,962, and 5,155,209, the contents of which are hereby incorporated by reference.

### Example 6

### Pharmacophore Modelling

Derivatives or analogues of exemplary compounds as shown in FIGS. 1 and 2 can be identified, designed, or predicted based on pharmacophore modeling. Pharmacophore structures of RecA inhibitors identified from the screen can be defined using various methods known in the art. For example, a structure of a RecA inhibitor selected from FIGS. 1 and 2 can be imported into and edited within CATALYST®. Pharmacophore can be defined by assembling the structural fragments with energy minimized to the closest local minimum using the CHARMM-like force field. Molecular flexibility is taken into account by considering each compound as an ensemble of conformers representing different accessible areas in a three dimensional space. The “best searching procedure” can be applied to select representative conformers within about 20 kcal/mol above the calculated global minimum. See Grigorov, M., et al. (1995) J. Chem. & Inf. Comp. Sci. 35:285-304, which is herein incorporated by reference. See Greene et al. (1994) J. Chem. Inf. & Comp. Sci. 34:1297-1308, which is herein incorporated by reference. Other methods for defining pharmacophore are available in the art.

Exemplary pharmacophore structures of RecA inhibitors identified from the screen are presented in FIG. 6. Additionally or alternatively, each exemplary pharmacophore structure shown in FIG. 6 can be further translated into coordinates by those skilled in the art.

Pharmacophore structures of the present invention are also intended to encompass any structures substantially similar to any one of the pharmacophores depicted in FIG. 6. As used herein, two pharmacophore structures are considered “substantially similar” if (1) they share at least 50% (e.g., at least 55%, at least 60%, at least 65%, at least 70%, at least 75%, at least 80%, at least 85%, at least 90%, at least 95%, at least 98%, or at least 99%) identical linkage bonds; (2) they share at least 50% (e.g., at least 55%, at least 60%, at least 65%, at least 70%, at least 75%, at least 80%, at least 85%, at least 90%, at least 95%, at least 98%, or at least 99%) identical atom coordinates defining the core structures, or equivalent structures having a root mean square deviation of less than about 5.0 Å(e.g., less than about 4.5 Å, less than about 4.0 Å, less than about 3.5 Å, less than about 3.0 Å, less than about 2.5 Å, less than about 2.0 Å, less than about 1.5 Å, less than about 1.0 Å); or (3) 50% (e.g., at least 55%, at least 60%, at least 65%, at least 70%, at least 75%, at least 80%, at least 85%, at least 90%, at least 95%, at least 98%, or at least 99%) identical atom coordinates defining surface-accessible features (e.g., hydrogen bond donors and acceptors, charged/ionizable groups, and/or hydrophobic patches) or equivalent features having a root mean square deviation of less than about 5.0 Å(e.g., less than about 4.5 Å, less than about 4.0 Å, less than about 3.5 Å, less than about 3.0 Å, less than about 2.5 Å, less than about 2.0 Å, less than about 1.5 Å, or less than about 1.0 Å).

Pharmacophore structures identified herein can be used to search various databases or virtual libraries using programs well known in the art to identify derivatives or analogues that may have similar or more potent RecA-binding or inhibitory activity. Exemplary programs suitable for the invention include, but are not limited to, the following:

1. GRID (Goodford, P. J. A Computational Procedure for Determining Energetically Favorable Binding Sites on Biologically Important Macromolecules. J. Med. Chem. 1985, 28, 849-857). GRID is available from Oxford University, Oxford, UK.
2. MCSS (Miranker, A.; Karplus, M. Functionality Maps of Binding Sites: A Multiple Copy Simultaneous Search Method. Proteins: Structure, Function and Genetics 1991, 11, 29-34). MCSS is available from Molecular Simulations, Inc., San Diego, Calif.
3. DOCK (Kuntz, I. D.; Blaney, J. M.; Oatley, S. J.; Langridge, R.; Ferrin, T. E. A Geometric Approach to Macromolecule-Ligand Interactions. J. Mol. Biol. 1982, 161, 269-288). DOCK is available from the University of California, San Francisco, Calif.

Once suitable binding orientations have been selected, complete molecules can be chosen for biological or pharmacological evaluation. In the case of molecular fragments, they can be assembled into a single inhibitor. This assembly may be accomplished by connecting the various moieties to a central scaffold. The assembly process may, for example, be done by visual inspection followed by manual model building, again using software such as Quanta or Sybyl. A number of other programs may also be used to help select ways to connect the various moieties. These include:

1. CAVEAT (P. A. Bartlett et al., “CAVEAT: A Program to Facilitate the Structure-Derived Design of Biologically Active Molecules,” in Molecular Recognition in Chemical and Biological Problems, Special Pub., Royal Chem. Soc., 78, pp. 182-196 (1989); G. Lauri and P. A. Bartlett, “CAVEAT: a Program to Facilitate the Design of Organic Molecules,” J. Comput Aided Ma Des., 8, pp. 51-66 (1994)). CAVEAT is available from the University of California, Berkeley, Calif.
2. 3D Database systems such as ISIS (MDL Information Systems, San Leandro, Calif.). This area is reviewed in Y. C. Martin. “3D Database Searching in Drug Design,” J. Med. Chem., 35, pp. 2145-2154 (1992).
3. HOOK (M. B. Eisen et al., “HOOK: A Program for Finding Novel Molecular Architectures that Satisfy the Chemical and Steric Requirements of a Macromolecule Binding Site,” Proteins: Struct., Funct. Genet., 19, pp. 199-221 (1994). HOOK is available from Molecular Simulations, San Diego, Calif.

A number of techniques commonly used for modeling drugs may be employed (For a review, see: Charifson, P. S., editor, Practical Application of Computer-Aided Drug Design, Marcel Dekker, Inc., 1997; Cohen, N. C.; Blaney, J. M.; Humblet, C.; Gund, P.; Barry, D. C., “Molecular Modeling Software and Methods for Medicinal Chemistry,” J. Med. Chem., 1990, 33, 883). There are likewise a number of examples in the chemical literature of techniques that can be applied to specific drug design projects. For a review, see: Navia, M. A. and Murcko, M. A., Current Opinions in Structural Biology, 1992, 2, 202. Some examples of these specific applications include: Baldwin, J. J. et al., J. Med. Chem., 1989, 32, 2510; Appelt, K. et al., J. Med. Chem., 1991, 34, 1925; and Ealick, S. E. et al., Proc. Nat. Acad. Sci. USA, 1991, 88, 11540.

Thus, combination of these steps can be used to design and predict novel RecA inhibitors that are useful in treating microbial infection and/or potentiating anti-microbial activity of other antibiotic agents.

### EQUIVALENTS AND SCOPE

Those skilled in the art will recognize, or be able to ascertain using no more than routine experimentation, many equivalents to the specific embodiments, described herein. The scope of the present invention is not intended to be limited to the above Description, but rather is as set forth in the appended claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any calcineurin substrate; any cell type; any neuronal cell system; any phosphosignature assay; any calcineurin modulator; any method of use; etc.) can be excluded from any one or more claims, for any reason, whether or not related to the existence of prior art.

### INCORPORATION OF REFERENCES

All publications and patent documents cited in this application are incorporated by reference in their entirety to the same extent as if the contents of each individual publication or patent document were incorporated herein.

What is claimed is:

1. A method comprising:
administering a RecA inhibitor having one of the structures shown in FIGS. 1 and 2, or a derivative thereof, to a subject suffering from or susceptible to a microbial infection; and
administering at least one antibiotic agent to the subject.

2. The method of claim 1, wherein the RecA inhibitor is administered in an amount effective to potentiate activity of the at least one antibiotic agent.

3.-4. (canceled)

5. The method of claim 1, wherein the RecA inhibitor is administered in an amount effective for suppression of resistance, such that resistance to the antibiotic agent occurs at a frequency below that observed under otherwise comparable conditions that lack RecA inhibitor administration.

6.-11. (canceled)

12. The method of claim 1, wherein the antibiotic agent is administered at a dose below its conventional dose.

13.-16. (canceled)

17. The method of claim 1, wherein the antibiotic agent is ciprofloxacin.

18. The method of claim 1, wherein the antibiotic agent is a gentamycin.

19. The method of claim 1, wherein the RecA inhibitor is Actinomycin D or a derivative thereof shown in FIG. 6.

20.-28. (canceled)

29. The method of claim 1, wherein the microbial infection is caused by bacteria that show resistance to at least one antibiotic, wherein the antibiotic is different from the antibiotic agent administered to the subject.

30.-31. (canceled)

32. A method comprising a step of:
administering a RecA inhibitor having one of the structures shown in FIG. 2, or a derivative thereof, to a subject suffering from or susceptible to a microbial infection.

33.-40. (canceled)

41. The method of claim 32, wherein the microbial infection is caused by bacteria that show resistance to at least one antibiotic.

42.-44. (canceled)

45. The method of claim 32, wherein the RecA inhibitor has the following structure: 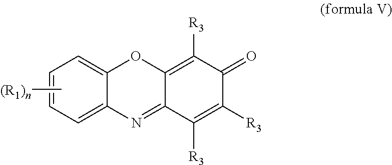
wherein
n is an integer between 0 and 4; inclusive;
each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —OC(O)R_{A}; —OC(O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; —OC(O)R_{C}; —OC(O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof.

46. A pharmaceutical composition comprising a RecA inhibitor having one of the structures shown in FIGS. 1 and 2, or a derivative thereof, and at least one antibiotic agent, wherein the pharmaceutical composition is formulated to treat microbial infection.

47. The pharmaceutical composition of claim 46, wherein the RecA inhibitor is present in an amount effective to potentiate activity of the at least one antibiotic agent.

48.-50. (canceled)

51. The pharmaceutical composition of claim 46, wherein the antibiotic agent is ciprofloxacin.

52. The pharmaceutical composition of claim 46, wherein the antibiotic agent is a gentamycin.

53. The pharmaceutical composition of claim 46, wherein the RecA inhibitor is Actinomycin D or a derivative thereof shown in FIG. 6.

54.-59. (canceled)

60. A compound of the formula (V): 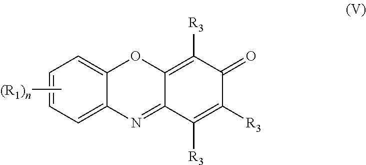
wherein
n is an integer between 0 and 4; inclusive;
each occurrence of R₁ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{A}; —C(═O)R_{A}; —CO₂R_{A}; —CN; —SCN; —SR_{A}; —SOR_{A}; —SO₂R_{A}; —NO₂; —N(R_{A})₂; —NHC(O)R_{A}; —OC(O)R_{A}; —OC(O)OR_{A}; or —C(R_{A})₃; wherein each occurrence of R_{A} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
each occurrence of R₃ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{C}; —C(═O)R_{C}; —CO₂R_{C}; —CN; —SCN; —SR_{C}; —SOR_{C}; —SO₂R_{C}; —NO₂; —N(R_{C})₂; —NHC(O)R_{C}; —OC(O)R_{C}; —OC(O)OR_{C}; or —C(R_{C})₃; wherein each occurrence of R_{C} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts thereof; wherein the compound is not of the formula: 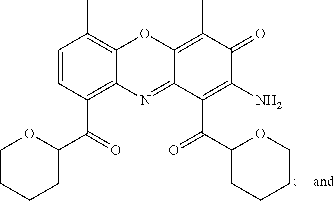
wherein the compound is not actinomycin D.

61. A pharmaceutical composition comprising a compound of claim 60, wherein the composition is formulated to treat an infectious disease.

62. A pharmaceutical composition of claim 61 further comprising another antibiotic agent.

63. A method of treating an infectious disease, the method comprising steps of:
administering a compound of claim 60 to a subject suffering from or susceptible to an infectious disease; and
administering at least one antibiotic agent to the subject.
